(19) United States
(12) Patent Application Publication
(10) Pub. No.: US 2022/0267252 A1
(43) Pub. Date: Aug. 25, 2022

(54) MESCALINE DERIVATIVES WITH MODIFIED ACTION

(71) Applicant: Mind Medicine, Inc., New York, NY (US)

(72) Inventors: Daniel TRACHSEL, Deltigen (CH); Matthias Emanuel LIECHTI, Oberwil (CH); Felix LUSTENBERGER, Meggen (CH)

(73) Assignee: Mind Medicine, Inc., New York, NY (US)

(21) Appl. No.: 17/676,169

(22) Filed: Feb. 20, 2022

Related U.S. Application Data
(60) Provisional application No. 63/153,317, filed on Feb. 24, 2021.

(51) Int. Cl. C07C 217/60; C07B 59/00; C07C 213/02

(52) U.S. Cl. CPC C07C 217/60; C07B 59/001; C07C 213/02; C07B 2200/05

(57) ABSTRACT

A composition for use in substance-assisted therapy, wherein: R is hydrogen, methyl, or ethyl, and R′ is C₁-C₅ branched or unbranched alkyl with the alkyl optionally substituted with F₁-F₅ fluorine substituents up to a fully fluorinated alkyl, C₃-C₆ cycloalkyl optionally and independently substituted with one or more substituents such as F₁-F₅ fluorine and/or C₁-C₂ alkyl, (C₃-C₆ cycloalkyl)-C₁-C₂ branched or unbranched alkyl optionally substituted with one or more substituents such as F₁-F₅ fluorine and/or C₁-C₂ alkyl, or C₂-C₅ branched or unbranched alkenyl with E or Z vinylic, cis or trans allylic, E or Z allylic or other double bond position in relation to the attached ether function, where any of the carbons of the branched or unbranched alkenyl substituent is optionally substituted independently with one or more C₁-C₂ alkyl, with F₁-F₅ fluorine or with D₁-D₅ deuteron substituents.

### BACKGROUND OF THE INVENTION

### 1. Technical Field

The present invention relates to both the substance definition and synthesis of novel mescaline analogs or derivatives to be used in substance-assisted psychotherapy.

### 2. Background Art

Psychedelics are substances capable of inducing exceptional subjective effects such as dream-like alterations of consciousness, affective changes, enhanced introspective abilities, visual imagery, pseudo-hallucinations, synesthesia, mystical-type experiences, disembodiment, and ego-dissolution (Liechti, 2017; Passie et al., 2008).

Efficacy data on the use of psychedelics for medical conditions have been reported for lysergic acid diethylamide (LSD) and addiction (Krebs & Johansen, 2012), LSD and anxiety associated with life-threatening illness (Gasser et al., 2014; Gasser et al., 2015), psilocybin and depression (Carhart-Harris et al., 2016a; Davis et al., 2020; Griffiths et al., 2016; Roseman et al., 2017; Ross et al., 2016), psilocybin and anxiety (Griffiths et al., 2016; Grob et al., 2011; Ross et al., 2016), and psilocybin and addiction (Bogenschutz, 2013; Bogenschutz et al., 2015; Garcia-Romeu et al., 2019; Garcia-Romeu et al., 2015; Johnson et al., 2014; Johnson et al., 2016). There is also evidence that the psychedelic brew Ayahuasca which contains the active psychedelic substance N,N-dimethyltryptamine (DMT) (Dominguez-Clave et al., 2016) may alleviate depression (de Araujo, 2016; Dos Santos et al., 2016; Palhano-Fontes et al., 2019; Sanches et al., 2016). In contrast, there are no comparable therapeutic studies or elaborated concepts on the use of the psychedelic substance mescaline or related substances to treat medical conditions.

Although no psychedelic is currently licensed for medical use, psilocybin and LSD are used already experimentally within clinical trials and special therapeutic-use programs (Andersson et al., 2017; Bogenschutz, 2013; Bogenschutz et al., 2015; Gasser et al., 2015; Griffiths et al., 2016; Grob et al., 2011; Krebs & Johansen, 2012; Ross et al., 2016; Schmid et al., 2020). Mescaline or its derivatives may be equally suitable to treat medical conditions. Specifically, existing psychedelic treatments such as LSD, psilocybin and DMT may not be suitable to be used in all patients considered for psychedelic-assisted therapy. The availability of several substances with different properties is important and the present lack thereof is a therapeutic problem which will further increase with more patients needing psychedelic-assisted therapy and an increase in demand for such treatment once the efficacy of first treatments will be documented in large clinical studies. For example, some patients may react with strong adverse responses to existing therapies such as psilocybin presenting with untoward effects including headaches, nausea/vomiting, anxiety, cardiovascular stimulation, or marked dysphoria.

Pharmacologically, mescaline is a phenethylamine unlike LSD and psilocybin. LSD, psilocybin, and mescaline are all thought to induce their acute psychedelic effects primarily via their common stimulation of the 5-HT2A receptor. All serotonergic psychedelics including LSD, psilocybin, DMT, and mescaline are agonists at the 5-HT2A receptor (Rickli et al., 2016) and may therefore produce overall largely similar effects. However, there are differences in the receptor activation profiles and in the subsequent signal transduction pathway activation patterns between the substances that may induce different subjective effects. LSD potently stimulates the 5-HT2A receptor but also 5-HT2B/C, 5-HT1 and D1-3 receptors. Psilocin, i.e., the active metabolite present in the human body derived from the prodrug psilocybin, also stimulates the 5-HT2A receptor but additionally inhibits the 5-HT transporter (SERT). Mescaline binds in a similar, rather low concentration range to 5-HT2A, 5-HT1A and α2A receptors. In contrast to LSD, psilocybin and mescaline show no affinity for D2 receptors. Taken together, LSD may have greater dopaminergic activity than psilocybin and mescaline, psilocybin may have additional action at the SERT. Mescaline and its derivatives do not interact with the SERT in contrast to psilocybin. Taken together the pharmacological profiles of LSD, psilocybin and mescaline show some differences but it is not clear whether these are reflected by differences in their psychoactive profiles in humans. Furthermore, mescaline has an old tradition of use but has not been compared with the more recently investigated psychedelics LSD and psilocybin and its therapeutic use potential has not been defined (Cassels & Saez-Briones, 2018).

In humans, subjective effects or psychoactive doses of mescaline appear within 30 minutes, peak at 4 hours and dose-dependently last 10-16 hours. The plasma half-life is approximately 6 hours (Charalampous, 1966). Mescaline is eliminated in urine mainly unchanged up to two thirds (⅔) of the dose ingested as well as the inactive metabolite 3,4,5-trimethoxyphenylacetic acid (TMPA) (Charalampous, 1966).

The acute subjective effects of psychedelics are mostly positive in most humans (Carhart-Harris et al., 2016b; Dolder et al., 2016; Dolder et al., 2017; Holze et al., 2019; Schmid et al., 2015). However, there are also negative subjective effects such as anxiety in many humans likely depending on the dose used, personality traits (set), the setting (environment) and other factors. The induction of an overall positive acute response to the psychedelic is critical because several studies showed that a more positive experience is predictive of a greater therapeutic long-term effect of the psychedelic (Garcia-Romeu et al., 2015; Griffiths et al., 2016; Ross et al., 2016). Even in healthy subjects, a more positive acute response to a psychedelic including LSD has been shown to be linked to more positive long-term effects on well-being (Griffiths et al., 2008; Schmid & Liechti, 2018).

Mescaline has relevant acute side effects to different degrees depending on the subject treated and including increased blood pressure, nausea and vomiting, negative body sensations, and dysphoria. Such side effects of a substance are often linked to its interactions with pharmacological targets. For example, interactions with adrenergic receptors may result in untoward clinical cardio-stimulant properties. Additionally, changes in the relative activation profile of serotonin 5-HT receptors change the quality of the psychoactive effects. Alterations in the binding potency, the binding mode, and the potency in activating the subsequent signaling pathways at 5-HT2A receptors may mostly determine the clinical dose to induce psychoactive effects. Alterations changing the metabolic stability of the compounds change the duration of action of the substance.

New mescaline derivatives are needed to provide substances with an improved effect profile such as, but not limited to, more positive effects, less adverse effects, different qualitative effects, and shorter or longer duration of acute effect.

### SUMMARY OF THE INVENTION

The present invention provides for a composition of a compound represented by FIG. 1 for use in substance-assisted therapy, wherein:

R is hydrogen, methyl, or ethyl, and

R′ is

C₁-C₅ branched or unbranched alkyl with the alkyl optionally substituted with F₁-F₅ fluorine substituents up to a fully fluorinated alkyl,

C₃-C₆ cycloalkyl optionally and independently substituted with one or more substituents such as F₁-F₅ fluorine and/or C₁-C₂ alkyl,

(C₃-C₆ cycloalkyl)-C₁-C₂ branched or unbranched alkyl optionally substituted with one or more substituents such as F₁-F₅ fluorine and/or C₁-C₂ alkyl, or

C₂-C₅ branched or unbranched alkenyl with E or Z vinylic, cis or trans allylic, E or Z allylic or other double bond position in relation to the attached ether function, where any of the carbons of the branched or unbranched alkenyl substituent is optionally substituted independently with one or more C₁-C₂ alkyl, with F₁-F₅ fluorine or with D₁-D₅ deuteron substituents.

The present invention provides a method of changing neurotransmission, by administering a pharmaceutically effective amount of a compound of FIG. 1 to a mammal, increasing serotonin 5-HT2A and 5-HT2C receptor interaction in the mammal, and inducing psychoactive effects.

The present invention also provides for a method of deuteration to obtain a compound represented by FIG. 1, by abstracting protons from the reacting molecule, such as, but not limited to, the compound 7 and its intermediates such as, but not limited to, compound 10a, covalently binding these initially abstracted protons in-situ, and quenching the resulting metalated difluorovinyl ether with a deuterium source.

### DESCRIPTION OF THE DRAWINGS

Other advantages of the present invention are readily appreciated as the same becomes better understood by reference to the following detailed description when considered in connection with the accompanying drawings wherein:

FIG. 1 shows the chemical structure of mescaline analogs or derivatives where R is hydrogen, methyl or ethyl; R′ is 1) C₁-C₅ branched or unbranched alkyl with the alkyl optionally substituted with F₁-F₅ fluorine substituents up to a fully fluorinated alkyl, 2) C₃-C₆ cycloalkyl optionally and independently substituted with one or more substituents such as F₁-F₅ fluorine and/or C₁-C₂ alkyl, 3) (C₃-C₆ cycloalkyl)-C₁-C₂ branched or unbranched alkyl optionally substituted with one or more substituents such as F₁-F₅ fluorine and/or C₁-C₂ alkyl, 4) C₂-C₅ branched or unbranched alkenyl with E or Z vinylic, cis or trans allylic, E or Z allylic or other double bond position in relation to the attached ether function, where any of the carbons of the branched or unbranched alkenyl substituent is optionally substituted independently with one or more C₁-C₂ alkyl, with F₁-F₅ fluorine or with D₁-D₅ deuteron substituents;

FIG. 2 exhibits illustrative examples (compounds 5a-5g and 6a-6g) of mescaline derivatives represented by FIG. 1 within the scope of invention;

FIG. 3 exhibits illustrative examples (compounds 5h-5m and 6h-6o) of mescaline derivatives represented by FIG. 1 within the scope of invention;

FIG. 4 exhibits illustrative examples (compounds 5r-5v, 6p-6q, 6u and 14) of mescaline derivatives represented by FIG. 1 within the scope of invention;

FIG. 5 summarily describes the synthetic route to the aldehydes 2a-2e; 2j-2s;

FIG. 6 summarily describes the synthetic route to the fluorinated vinylether-containing aldehydes 2f and 2g;

FIG. 7 summarily describes the synthetic route to the deuterofluorinated vinylether-containing aldehydes 2h and 2i;

FIG. 8 summarily describes the synthetic route to the aldehydes 2t-2v;

FIG. 9 summarily describes the synthetic route to produce homoscalines 5a-m and 5r-5v as well as to the 3C-homoscalines 6a-6q and 6u, starting from the aldehydes 2a-v, via the nitroolefines 3a-m and 3r-3v as well as 4a-4q and 4u; and

FIG. 10 summarily describes the synthetic route to produce homoscaline 14, starting with homoscaline 5t.

### DETAILED DESCRIPTION OF THE INVENTION

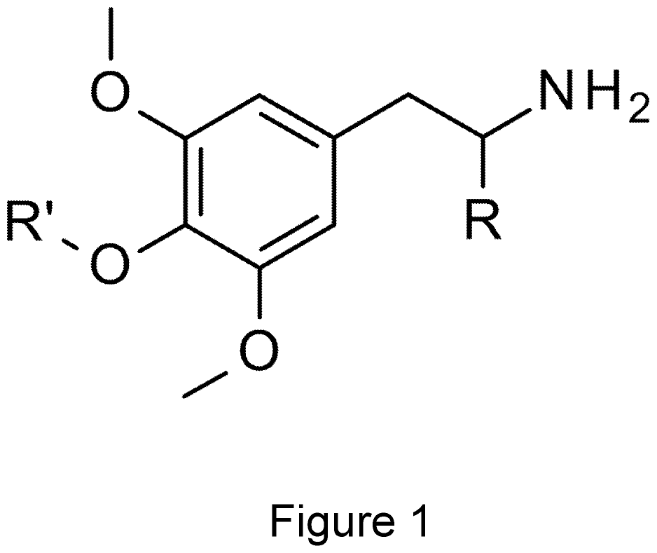

The present invention provides for mescaline derivatives. More specifically, the present invention provides for a composition of a compound represented by FIG. 1 for use in substance-assisted therapy, wherein:

R is hydrogen, methyl, or ethyl, and

R′ is

C₁-C₅ branched or unbranched alkyl with the alkyl optionally substituted with F₁-F₅ fluorine substituents up to a fully fluorinated alkyl,

C₃-C₆ cycloalkyl optionally and independently substituted with one or more substituents such as F₁-F₅ fluorine and/or C₁-C₂ alkyl,

(C₃-C₆ cycloalkyl)-C₁-C₂ branched or unbranched alkyl optionally substituted with one or more substituents such as F₁-F₅ fluorine and/or C₁-C₂ alkyl, or

C₂-C₅ branched or unbranched alkenyl with E or Z vinylic, cis or trans allylic, E or Z allylic or other double bond position in relation to the attached ether function, where any of the carbons of the branched or unbranched alkenyl substituent is optionally substituted independently with one or more C₁-C₂ alkyl, with F₁-F₅ fluorine or with D₁-D₅ deuteron substituents.

The compounds represented by FIG. 1 are basic compounds which form acid addition salts with inorganic or organic acids. Therefore, they form pharmaceutically acceptable inorganic and organic salts with pharmacologically acceptable inorganic or organic acids. Acids to form such salts may be selected from inorganic acids such as hydrochloric acid, hydrobromic acid, hydroiodic acid, sulfuric acid, nitric acid, phosphoric acid, and the like, and organic acids, such as carbonic acid, p-toluenesulfonic acid, methanesulfonic acid, oxalic acid, succinic acid, citric acid, benzoic acid, and the like. Examples of such pharmaceutically acceptable salts thus are the sulfate, pyrosulfate, bisulfate, sulfite, bisulfite, phosphate, monohydrogen-phosphate, dihydrogenphosphate, metaphosphate, pyro-phosphate, chloride, bromide, iodide, formate, acetate, propionate, decanoate, caprylate, acrylate, isobutyrate, caproate, heptanoate, oxalate, malonate, succinate, suberate, sebacate, fumarate, maleate, benzoate, phthalate, sulfonate, phenylacetate, citrate, lactate, glycollate, tartrate, methanesulfonate, propanesulfonate, mandelate and the like. Preferred pharmaceutically acceptable salts are those formed with hydrochloric acid.

The general chemical terms used for the FIG. 1 have their usual meanings. For example, the term “alkyl” includes such groups as methyl, ethyl, n-propyl, isopropyl, n-butyl, isobutyl, sec-butyl, tert-butyl, and the like. For another example, the term “cycloalkyl” includes such groups as cyclopropyl, cyclobutyl, cyclopentyl, and the like. Further on, the term “alkenyl” includes such groups as vinyl (ethenyl), 1-propenyl, 2-propenyl, isopropenyl, butenyl, and the like.

Those skilled in the art will appreciate that certain of the compounds of the present invention have at least one chiral carbon, and may therefore exist as a racemate, as individual enantiomers or diastereomers, and as mixtures of individual enantiomers or diastereomers in any ratio. For example, individual enantiomers of compounds of the invention are illustrated in FIG. 1 where R is Me or Et. Those skilled in the art will also appreciate that those compounds of the invention where R′ in FIG. 1 consists of a chiral substituent, will bear an additional asymmetric center which create additional optical isomers as described above. While it is a preferred embodiment of the invention that the compounds of the invention exist are used as racemates or mixtures of diastereomers, the present invention also contemplates the compounds of the invention existing in individual enantiomeric or diastereomeric pure form.

The individual enantiomers and diastereomers may be prepared by chiral chromatography of the racemic or enantiomerically or diastereomerically enriched free amine, or fractional crystallization of salts prepared from racemic- or enantiomerically- or diastereomerically-enriched free amine and a chiral acid. Alternatively, the free amine may be reacted with a chiral auxiliary and the enantiomers or diastereomers separated by chromatography followed by removal of the chiral auxiliary to regenerate the free amine. Furthermore, separation of enantiomers or diastereomers may be performed at any convenient point in the synthesis of the compounds of the invention. The compounds of the invention may also be prepared by application of chiral syntheses. The compound itself is a pharmacologically acceptable acid addition salt thereof.

In patients that have adverse reactions to other psychedelics, mescaline-like substances can be useful as alternative treatments. In some patients, mescaline derivatives can also be useful because another experience than made with psilocybin or LSD is necessary or because a patient is not suited for therapy with these existing approaches a priori. Thus, mescaline derivatives of FIG. 1 can serve as alternative treatment options with characteristics sufficiently similar to other psychedelics to be therapeutic but also sufficiently different to provide added benefits or avoid negative effects of other psychedelics.

Based on structural similarities, the compounds of FIG. 1 described in the present invention are expected to have overall similar pharmacological properties as mescaline as described above.

The present invention provides compounds of FIG. 1 that are pharmacologically active and allow changing the neurotransmission and/or producing neurogenesis. More specifically, but not excluding, the compounds interact with serotonin (5-HT, 5-hydroxytryptamine) 5-HT2A and 5-HT2C receptors in mammals by administering to a mammal in need of such interaction a pharmaceutically effective amount of a compound of FIG. 1.

Therefore, the present invention provides a method of changing neurotransmission, by administering a pharmaceutically effective amount of a compound of FIG. 1 to a mammal, increasing serotonin 5-HT2A and 5-HT2C receptor interaction in the mammal, and inducing psychoactive effects.

The neuronal interaction of compounds represented in FIG. 1 can be used in mammals for substance-assisted psychotherapy where the compounds induce psychoactive effect to enhance psychotherapy. The preferred mammal is human.

The intensity and quality of the psychoactive effect including psychedelic or empathogenic effects, the quality of perceptual alterations such as imagery, fantasy and closed or open eyes visuals, and body sensation changes, the pharmacologically active doses, may be similar or different to that of the original molecule mescaline.

Not only receptor interactions may change by structural modifications represented in FIG. 1 but also the metabolism can be modified significantly by making a rather labile vinyl ether compound more or less prone to metabolism by introducing alkyl groups, fluorine atoms and deuterium atoms to this functional group in either vinyl, allyl or gamma positions, as aforementioned. Thus, the invention allows for the synthesis of psychedelic compounds with a relatively shorter duration of action compared to the more metabolically stable and longer-acting parent compound.

The structure of 4-O alkyl-analogs of mescaline described herein were previously described by Shulgin and others (Shulgin & Shulgin, 1991), including also two O-alkenyls, one O-alkyne, one O-(cycloalyl)alkyl derivative, one O-benzyl, one O-phenethyl. Only the structures were described and some acute effect data including duration of action and doses (Shulgin & Shulgin, 1991), not the pharmacological profiles and human therapeutic uses.

Two 4-O-substituted analogs of mescaline, namely the 4-butyloxy and the 4-benzyloxy analog (Basel, 1932) have been patented as substances and for a non-specified “therapeutic use” in Switzerland in the 1930s.

Trachsel (Trachsel, 2002) preliminarily described the synthesis of seven 3C scalines (FIG. 1: R=Me; including O-alkyls, O-fluoroalkyls, and O-alkenyls) without information on pharmacology or human use.

Trachsel (Trachsel et al., 2013) described 5-HT2A and 5-HT2C receptor binding data of the above compounds but no other profiling data. Additional profiling data has now also been published after the filing of the present provisional patent application (Kolaczynska et al., 2022). Additionally, the same 5-HT data and qualitative reaction schemes were given for CP, V, DFIP, TFP, DFM, 3C-DFM and TFM.

Furthermore, a series of mescaline derivatives of FIG. 1 never described in any way were newly synthesized within the present invention. These include compounds with R′=vinyl groups, cycloalkyl groups directly attached to the 4-O function, fluorinated alkenyl substituents and deuterated fluorinated alkenyl substituents. The derivatives of mescaline represented in FIG. 1 are expected to act similarly as mescaline with some modified action.

Derivatives of mescaline can include 3-alkoxy substitution variations or 4-alkoxy substitution variations of the phenethylamine structure forming “scalines” or may include the addition of the methylation of the alpha carbon of the phenethylamine structure to form amphetamines also containing the above 3,4,5-substitutions on the phenyl ring to form “3C-scalines” (Shulgin & Shulgin, 1991; Trachsel et al., 2013). Several previously described (Trachsel et al., 2013) and new such mescaline derivatives represented in FIG. 1 were newly synthesized in the present invention. The presently synthesized derivatives include 4-O-alkyls, 4-O-cycloalkyls, 4-O-fluoroalkyls, 4-O-fluoroalkenyls and O-alkenyls and deuterated forms of the aforementioned ones and no 4-S-derivatives which are also known but not described herein.

While all the mescaline derivatives represented in FIG. 1 are useful in optimizing the clinical effect profile of mescaline, certain classes of the compounds are preferred, such as wherein the compound is a free base, a salt, a hydrochloride salt, a racemate where applicable, a single enantiomer, a single diastereomer, or a mixture of enantiomers or diastereomers in any ratio. It will be understood that these classes can be combined to form additional preferred classes.

A general strategy to access some of the compounds of the field of invention is known. The O-alkylation of syringaldehyde (Shulgin & Shulgin, 1991; Trachsel, 2002) by using calcium carbonate, sodium iodide and an alkylating agent in dimethyl sulfoxide has been described before. The preparation of the nitroolefins from these O-alkylated syringaldehydes by the reaction with nitromethane or nitroethane, generally referred as the Henry reaction, has been described and was mostly catalyzed by alcoholic solution of sodium or potassium hydroxide (Basel, 1932) or ammonium acetate (Shulgin & Shulgin, 1991), or n-butylamine and acetic acid (Trachsel, 2002). The nitroolefins are reduced to the corresponding scalines or 3C-scalines by using lithium aluminum hydride (LAH) or alane generated in situ from LAH and concentrated sulfuric acid (Trachsel, 2002). Another approach allowing to access the final scaline (but not 3C-scaline) compounds is the formation of a methiodide of a (dimethylaminomethyl)phenol, treating it with potassium cyanide to access the corresponding phenyl acetonitrile and either reducing it or further 4-O-alkylating it and then reducing it to the final scaline (Shulgin & Shulgin, 1991).

The present invention can further optimize the Henry reaction for achieving higher yields, applying lower reaction temperatures, e.g., 60° C. vs. 110° C., and for shorter reaction times needed (usually <1 h vs. numerous hours) as well as for the use of much less of the nitroalkane (approx. 2-2.5 mass equivalents vs. 5-10 mass equivalents). This could be achieved by using catalytic amounts of a combination of n-butylamine and acetic acid and an eventual combination of the addition of small amounts of molecular sieves to the reaction mixture.

Accessing simple fluorinated vinyl ethers by dehydrofluorination and trapping them with water or methyl iodide has been described in 1976 (Nakai et al., 1976). A deuterated form has also been mentioned by the same authors although their procedure describes a success rate of deuteration of only approx. 8.6:1 on a lithiated difluoro-vinyl ether.

The present invention can enhance the previously mentioned extent of deuteration significantly, i.e., one order of magnitude, in trapping the two protons initially being abstracted by lithium diisopropylamide from the reacting molecule, e.g., a 2,2,2-trifluoroethoxy ether, by in-situ binding them covalently to the butane anions by adding two equivalents of butyl lithium to the reaction mixture, before quenching the lithiated difluoro-vinyl ether with deuterium oxide. By such, the two protons initially bound to two molecules diisopropylamine are permanently removed from the reaction mixture and cannot anymore exchange with any deuterium oxide entering the reaction mixture prior reaction with the lithiated difluoro-vinyl ether or with deuteroxide anions formed after initial reaction with the lithiated difluoro-vinyl ether. With this modified procedure the present invention reached deuteration ratios of 99:1.

This achievement in high deuteration rate is of great importance since well-defined deuterium levels are required to have a defined kinetic isotope effect in relation to drug dose and drug effect in a patient. Furthermore, deuterium atoms can greatly affect the metabolic stability of a molecule and thus play an important role in the overall action of such a compound.

The group presented in the preparation section, namely compounds 5a to 5m, 5r to 5v, 6a to 6q, 6u and 14, is illustrative of mescaline derivatives represented in FIG. 1 contemplated within the scope of the invention.

In order to have well-defined deuterated analogs available, a modified high yield deuteration reaction was invented.

Therefore, the present invention also provides for a method of deuteration to obtain a compound represented by FIG. 1, by abstracting protons from the reacting molecule, such as, but not limited to, the compound 7 and its intermediates such as, but not limited to, compound 10a, covalently binding these initially abstracted protons in-situ, and quenching the resulting metalated difluorovinyl ether with a deuterium source. The abstracting protons step can be achieved by adding a deprotonating agent (such as, but not limited to diisopropylamides, tert-butoxides, bis(trimethylsilyl)amides, or a tetramethylpiperidide (such as, but not limited to lithium, sodium, or potassium)). The covalently binding step is achieved by adding a reagent such as butyl lithium or methyl lithium. The deuterium source of step 3) can be D2O or a deuterated alcohol.

Several of the synthesized scalines and their amphetamine congeners were investigated at key targets in vitro (data published after filing (Kolaczynska et al., 2022)). The main target of psychedelics is the 5-HT2A receptor (Holze et al., 2020) and typically there is a high affinity binding at this receptor (Rickli et al., 2016). Additionally, the binding potency at the 5-HT2A receptor is typically predictive of the human doses of psychedelics to be psychoactive for many compounds (Luethi & Liechti, 2018). Furthermore, the psychedelic effects of psilocybin in humans have been shown to correlate with 5-HT2A receptor occupancy measures using positron emission tomography (Madsen et al., 2019). Thus, interactions with this target are relevant and predict psychedelic action with high likelihood for most psychedelics. However, this may not be the case for all substances within this class.

Additional receptors such as the serotonergic 5-HT1A and 5-HT2C or dopaminergic D2 receptors are thought to moderate the effects of psychedelics (Rickli et al., 2016). Although some psychedelics like psilocybin do not directly act on dopaminergic receptors, they have nevertheless some dopaminergic properties by releasing dopamine in the striatum (Vollenweider et al., 1999) likely via 5-HT1A receptor activation (Ichikawa & Meltzer, 2000). Furthermore, LSD has activity at D2 receptors (Rickli et al., 2016) and some of its behavioral effect may be linked to this target (Marona-Lewicka et al., 2005).

Activity of compounds at monoamine transporters are thought to mediate MDMA-like empathogenic effects (Hysek et al., 2012). Importantly, mescaline is a very weak 5-HT2A receptor ligand and high doses are needed to induce psychoactive effects in humans. However, despite its low potency, mescaline can have extraordinarily strong psychedelic effects in humans at high doses and the same is likely the case for the substances developed within the present invention although 10-20-fold higher potency is also possible in some compounds, to be evaluated in detail clinically. Key results of pharmacological profiling of the compounds described herein were:

Most mescaline derivatives represented in FIG. 1 showed binding affinity and agonistic activity at the serotonin 5-HT2A receptor indicating activity as psychedelics. The binding potency was generally low similar to mescaline with a few exceptions and lower than that of psilocin and much lower than that of LSD and consistent with a need for higher mg doses of mescaline and its derivatives to induce psychedelic effects in humans.

There were marked differences among the mescaline derivatives represented in FIG. 1 regarding binding potency at the 5-HT receptors, relative binding potency with regards to 5-HT_{2A} over 5-HT₁ or over 5-HT_{2C} receptor binding, as well as some differences regarding binding to adrenergic α₂ receptors. In contrast to LSD, mescaline and its derivatives did not relevantly bind to dopaminergic receptors. In contrast to psilocybin which is a moderate SERT inhibitor, mescaline and its derivatives did not inhibit monoamine transport.

Together, the in vitro profiles of mescaline and its derivatives represented in FIG. 1 compared with that of psilocin and LSD indicate overall psychedelic properties of all compounds but also differences that likely manifest when used in humans. Accordingly, some mescaline derivatives will exert psychedelic acute effect profiles that are more beneficial to some patients including but not limited to: more overall positive effects, more or less perceptual effects, more emotional effects, less anxiety, less cardio-stimulant effects, less adverse effects, less nausea, longer and also shorter effects among other properties and compared to mescaline. Specifically, taken together the pharmacological data and structural specifics on the substances tested herein some compounds are of particular interest. FE and FP have a relatively short duration of action (<6 hours) compared with mescaline and potentially empathogenic MDMA-like effects. DFM and TFE are relatively potent, longer acting (12-18 hours) and having psychedelic properties.

There are several problems when using mescaline that can be solved using the compounds described herein. Namely, high doses of mescaline (200-800 mg) are needed to induce a full psychedelic experience. Derivatives represented in FIG. 1 can be more potent resulting in reduced need of the substance. Psychedelics like psilocybin produce adverse effects including nausea and vomiting, cardiovascular stimulation, and an increase in body temperature and others. The novel compounds produce less nausea, less cardio stimulation, less thermogenesis and/or other adverse responses. Mescaline has a long duration of action. The presently developed substances were designed to have similar qualitative effects to mescaline while acting shorter or to have a long duration of action but other qualitative effects as reflected by their structural changes and associated pharmacological properties. In particular, metabolically less-stable compounds were created to shorten the plasma half-life and duration of action in humans. Other alterations of the chemical structure were designed to create substances with qualitative effects different from those of mescaline and creating subjective effects that are considered beneficial to assist psychotherapy including feelings of empathy, openness, trust, insight, and connectedness and known to those knowledgeable in the field.

The compounds represented by FIG. 1 act with shorter, with similar or with longer duration of action in human in comparison to the original mescaline molecule. This is triggered by modification of the molecular structure in FIG. 1.

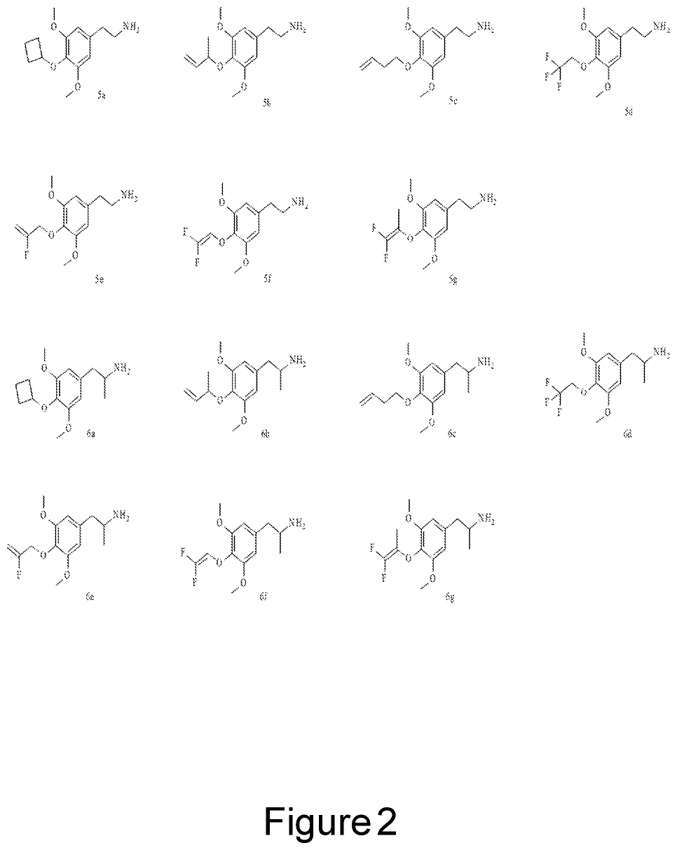

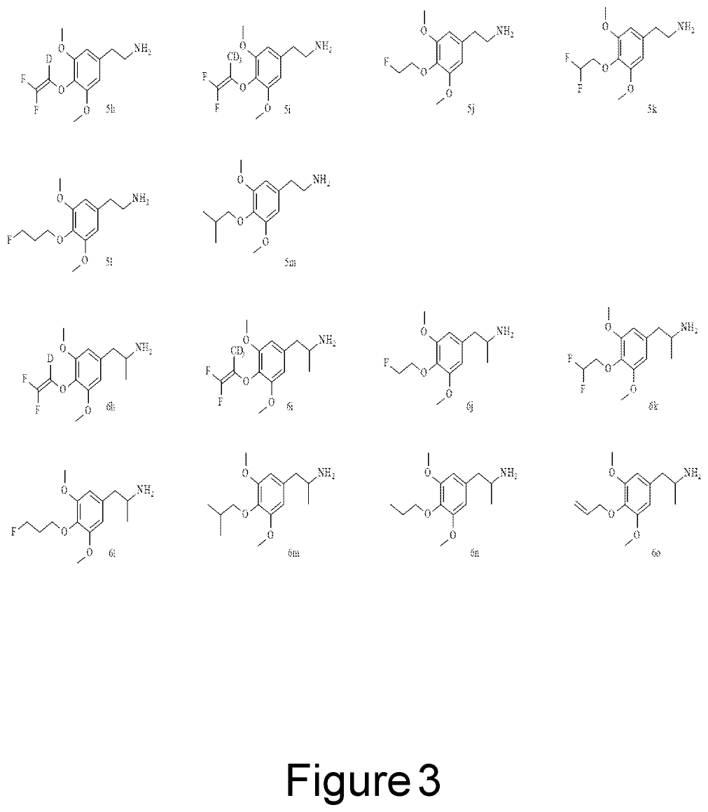

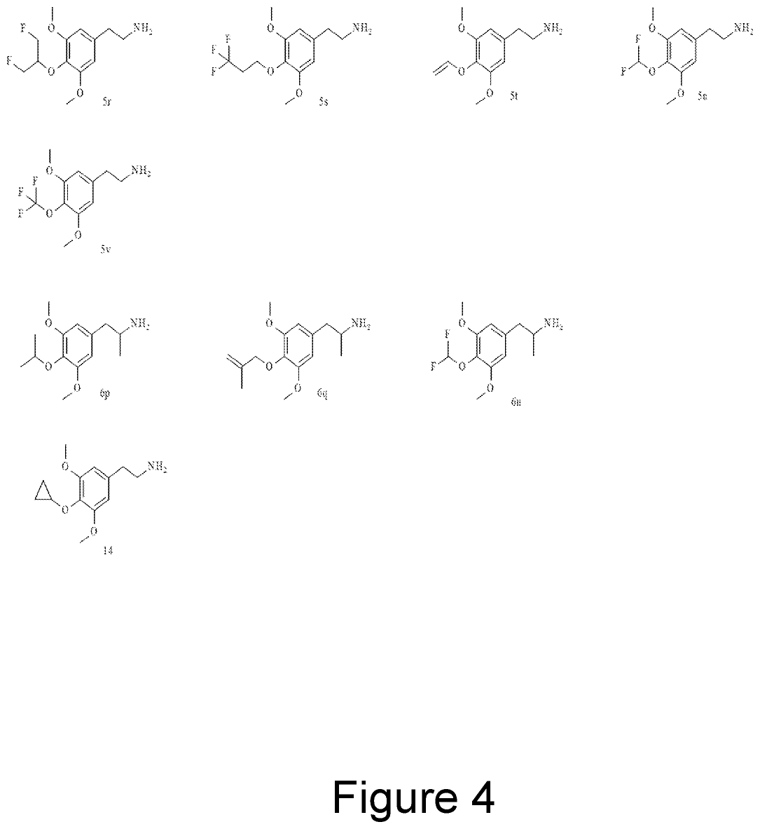

The group presented in the preparation section, namely compounds 5a to 5m, 5r to 5v, 6a to 6q, 6u and 14 (chemical structures see FIGS. 2-4), is illustrative of mescaline derivatives represented in FIG. 1 contemplated within the scope of the invention.

In order to have well-defined deuterated analogs available, a modified and high yield deuteration rate reaction was invented.

The invented compounds represented in FIG. 1 allow modification of the mode of action, the psychodynamic processes, and the qualitative perceptions, e.g., in terms of psychedelic or empathogenic intensity in comparison to the original mescaline molecule.

The invented compounds represented in FIG. 1 may cause similar or different quality of imagery, fantasy and closed or open eyes visuals in comparison to the original mescaline molecule.

The invented compounds represented in FIG. 1 may have a similar or a higher dose potency in comparison to the original mescaline molecule.

The invented compounds represented in FIG. 1 may cause similar or more favorable body feelings in comparison to the original mescaline molecule.

The aforementioned characteristics can be modified in a progressive way by the introduction of one or more fluorine atoms, by one or more deuterium atoms and by one or more alkyl groups, independently or in any combination, to the alkenyl group in either vinyl, allyl or further isolated positions.

The modified properties can be tailored and applied individually to the patient's need. This is not only targeted by changing the compound's receptor profile but also greatly by the modification of ADME (Absorption, Distribution, Metabolism and Excretion) via the introduction of more, similar or less liable 4-O substituents in compounds represented in FIG. 1.

Preparations of the Compounds

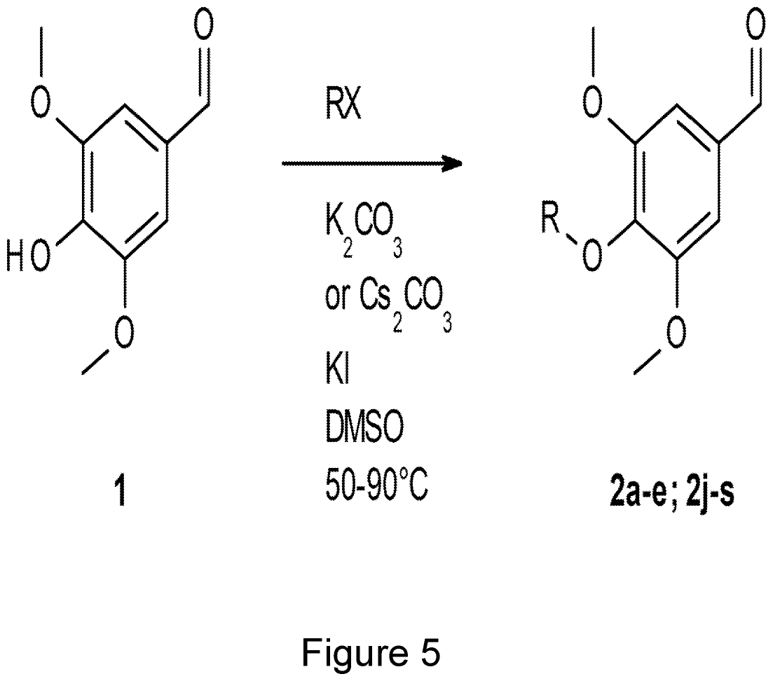

The general access to the homoscalines and 3C-homoscalines is outlined in FIGS. 5 to 10. The commercially available syringaldehyde is converted to the corresponding 4-O-alkylated aldehydes (such as illustrated in FIG. 5, compounds 2a-e and 2j-s) by using an appropriate base such as, but not limited to alkali bases, alkali carbonates such as calcium carbonate or cesium carbonate, no catalyst or a catalyst such as potassium iodide, an appropriate solvent with branched or unbranched carbon chain lengths of C1-C6 such as an alcohol, ketone, dimethyl formamide, diethyl formamide, dimethyl sulfoxide, tetrahydrofuran with or without the addition of water and an alkylating or fluorinated alkylating agent such as branched or unbranched cyclic or non-cyclic alkyl or alkenyl halides, alkyl sulfonates and any fluorinated sulfonates such as triflates. The temperature may range from 0-150° C., more favorably 20-100° C.

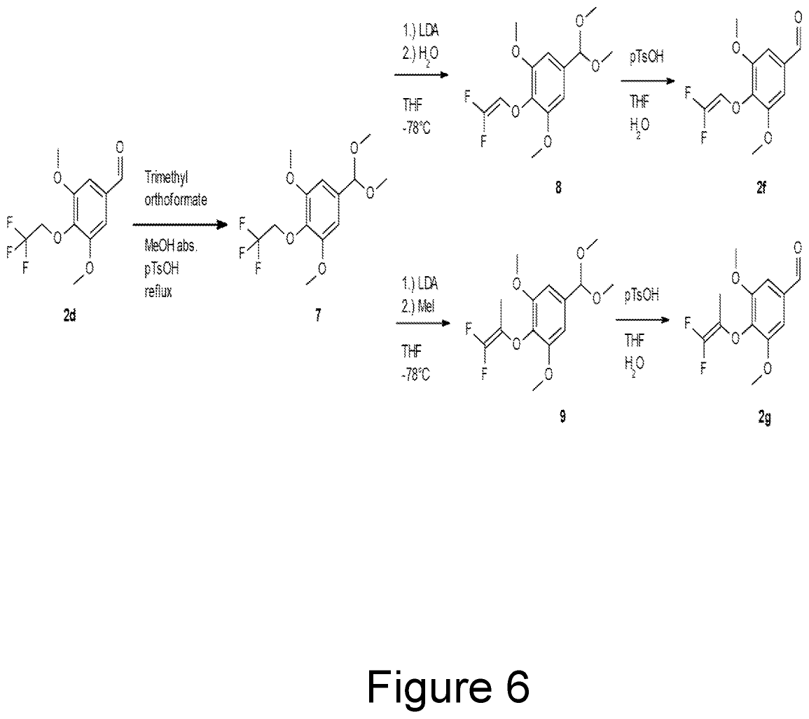

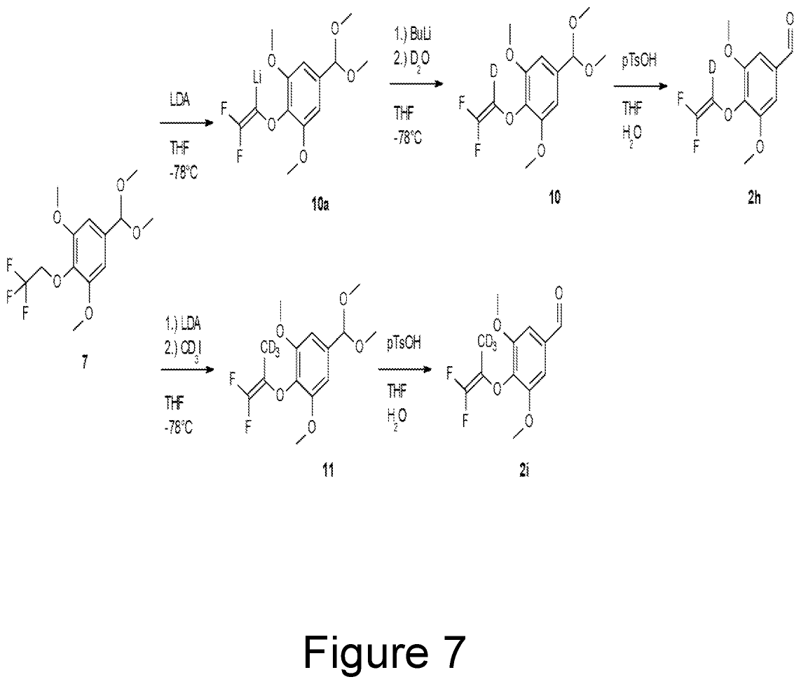

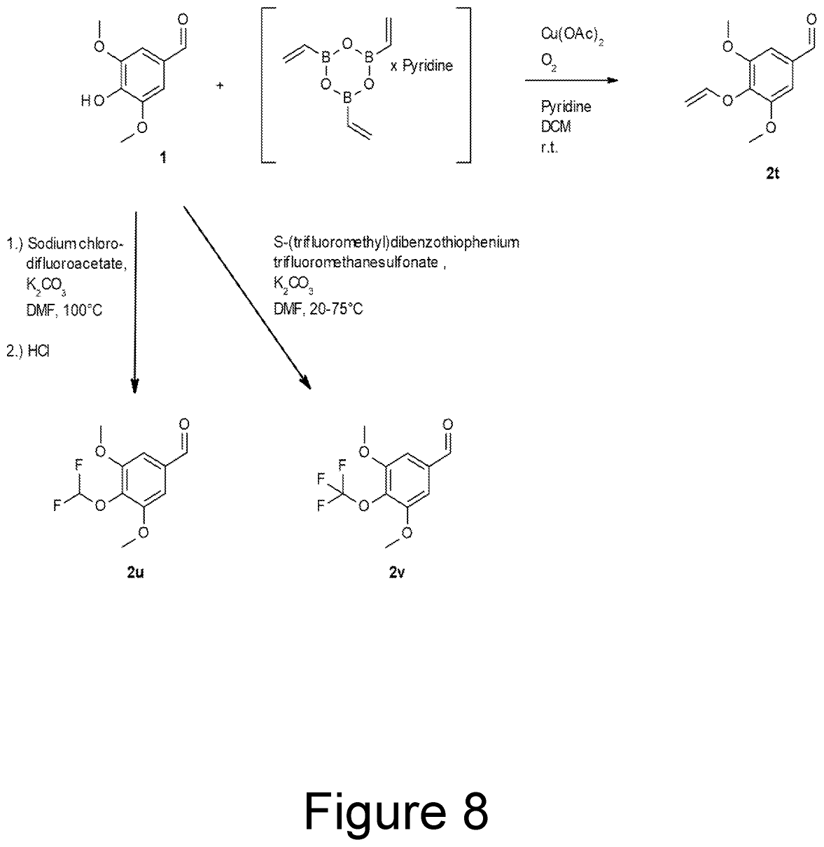

The corresponding aldehydes containing 4-vinyl ethers and substituted 4-vinyl ethers may be accessed by either reaction of syringaldehyde with corresponding trivinylcyclotriboroxane-pyridine complexes (such as illustrated in FIG. 8) according to (McKinley & O'Shea, 2004). Corresponding aldehydes containing fluorinated 4-vinyl ethers and additionally substituted fluorinated 4-vinyl ethers (such as illustrated in FIG. 6) may be accessed by 4-O-alkylating syringaldehyde with a branched or unbranched fluorinated alkyl or alkenyl halide under conditions described before, and then protecting the carbaldehyde function to a functional group being inert to strong bases such as diisopropylamides, tert-butoxides, bis(trimethylsilyl)amides or tetramethylpiperidides of lithium, sodium, or potassium. The protected aldehyde derivative is then treated with such a base at a favorable temperature such as below 0° C. or more favorably −50° C. and most favorably at below −70° C. allowing to selectively dehydrohalogenate at the 4-O-alkyl substituent to the corresponding fluorinated 4-O-vinyl ethers (such as illustrated in FIG. 6). By applying sufficient of any of the mentioned bases, the dehydrohalogenated fluorinated 4-O-vinyl ethers are allowed further to deprotonate in the vinyl position and can be trapped with water, deuterated water or another deuteron donor such as deuterated methanol, or an alkylating agent such as a branched or unbranched non-deuterated or deuterated alkyl halide or sulfonate or triflate, as illustrated in FIG. 6 and FIG. 7. In case of quenching the further deprotonated vinyl intermediate with a deuteron source such as deuterated water or deuterated methanol, the formerly abstracted protons are bound covalently preferably by adding sufficient butyl lithium, methyl lithium, or any other suitable metalated organic compound prior the deuteriation process, as illustrated in FIG. 7. With that, the obtained carbaldehyde-protected fluorinated 4-O-vinyl ethers or any deuterated form thereof can then be deprotected by suitable conditions to get the desired aldehydes, as illustrated in FIG. 6 and FIG. 7. These may include, but not be limited to acidic conditions such as p-toluenesulfonic acid (pTsOH), hydrochloric acid or trifluoroacetic acid or allyl bromide in an appropriate solvent with branched or unbranched carbon chain lengths of C₁-C₆ such as an alcohol, ketone, dimethyl formamide, diethyl formamide, dimethyl sulfoxide, tetrahydrofuran, chlorinated alkanes with or without the addition of water, acetone, alcohol, an alicyclic or cyclic ether or a mixture thereof.

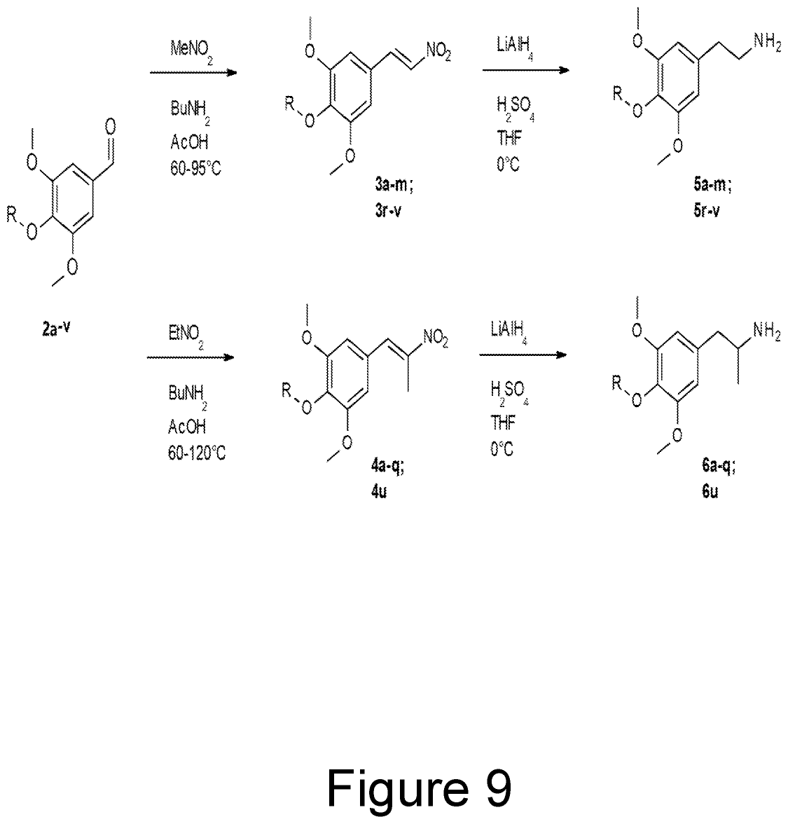

The 4-O-alkylated 3,5-dimethoxybenzaldehydes are then subjected to an aldol condensation, namely the Henry reaction, by mixing any of these aldehydes with a nitroalkane such as nitromethane, nitroethane or 1-nitropropane and a catalyst such as an organic salt or a mixture of an organic base and an organic acid, most favorably n-butylamine and acetic acid (such as illustrated in FIG. 9). The mixture may or not then be treated with heat in absence or presence of a drying agent such as an inorganic salt or, most favorably, molecular sieves. The water formed may also be removed azeotropically during reaction. The reaction mixture may be cooled, and the product solids formed may be filtered of, or the mixture may be concentrated in vacuo prior further treatment. The obtained residue may be further purified by crystallization or recrystallization or by column chromatography in order to get the final nitroolefines such as 3a-m and 3r-3v as well as 4a-4q and 4u as illustrated in FIG. 9.

As such, the obtained nitroalkenes are dissolved in an inert solvent such as tetrahydrofuran or diethyl ether and added to a suspension of alane generated in situ from allowing to react lithium aluminum hydride (LiAlH₄) with concentrated sulfuric acid (H₂SO₄) in a similar solvent (such as illustrated in FIG. 9). The reaction temperature may be set between −20° C. and 70° C., favorably at 0° C.-60° C. The reaction mixture is then quenched subsequently with an alcohol, favorably isopropanol, and then with a base such as aqueous sodium hydroxide before filtering it off. The Filtrate is concentrated in vacuo and during the process an inert gas such as argon or nitrogen may be applied in order to prevent any carbamate formation. The residual scaline or 3C-scaline free base (such as of 5a-m and 5r-5v as well as of 6a-6q and 6u, as illustrated in FIG. 9) is then dissolved in a solvent, favorably non-protic, most favorably in diethyl ether or dioxane, and neutralized by the addition of anhydrous hydrogen chloride or sulfuric acid or any other salt forming organic agent such as fumaric acid, tartaric acid, or acetic acid in a similar solvent.

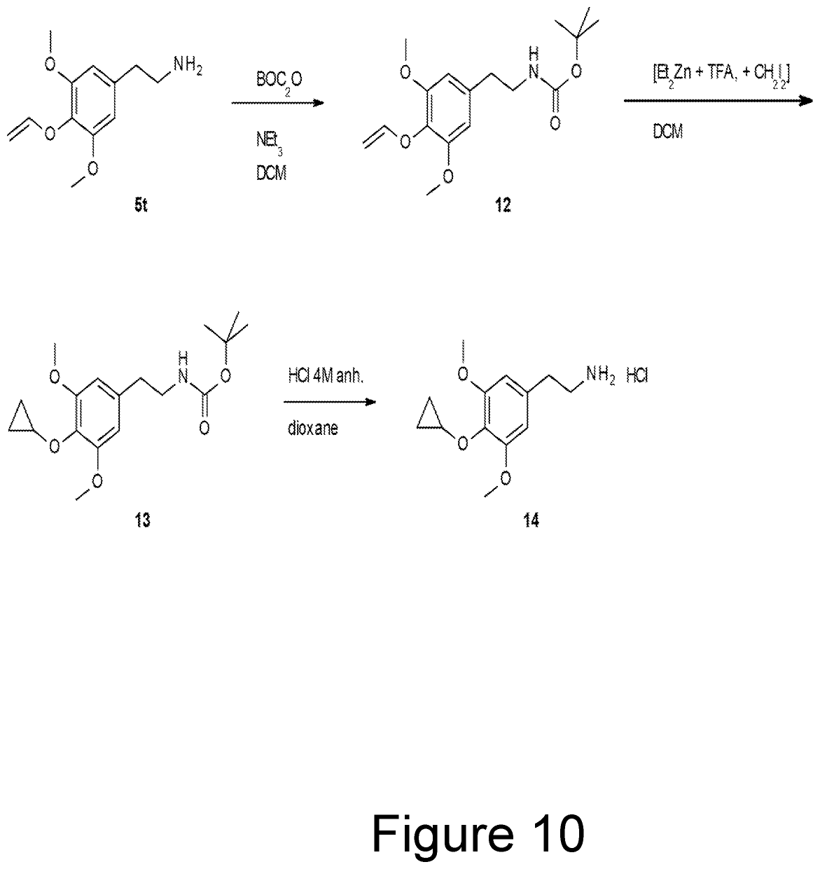

In order to access the cyclopropyl derivatives such as represented by compound 14 (illustrated in FIG. 10), the compound is prepared from the corresponding vinyl ether derivative by a cyclopropanation reaction via the Simmons-Smith reaction on an appropriately N-protected derivative. Such protecting groups may be t-butoxycarbonyl or any other conditions-resistant group. To access the final compound the protecting group is removed by known procedures.

Detailed Description of the Chemical Preparation of the Compounds

General method for the 4-O-alkylations. To a solution of syringaldehyde in dimethyl sulfoxide (DMSO) anhydrous (anh.) is added potassium iodide and potassium carbonate or cesium carbonate under an inert atmosphere. The well stirred mixture is placed in a preheated heating bath at 85° C. Next, the alkyl halide is added quickly. Stirring becomes progressively better over time. When the reaction is complete (monitoring by thin-layer chromatography (TLC): dichloromethane) the mixture is poured into ice-water and extracted three times with dichloromethane. The combined organic extracts are successively washed with 2×NaOH 2M, with 3× water and once with brine, dried over sodium sulfate and concentrated in vacuo to get the desired 4-O-alkylated syringaldehyde.

General method for the nitro olefination (modified Henry reaction). The 4-O-alkylated syringaldehyde is dissolved in nitromethane or nitroethane under slight warming. Next, molecular sieves 3A (where applied), n-butylamine and acetic acid is added, and the mixture is gently stirred at 60-110° C. under an inert atmosphere. When the reaction is complete (monitoring by TLC, i.e., dichloromethane) the mixture is separated from the molecular sieves and concentrated in vacuo. The residue is either recrystallized from an appropriate solvent or purified by dissolving it in a small amount of organic solvent and eluting it with organic solvent through a short path silica gel column. The eluate obtained is concentrated in vacuo.

General method for the alane-promoted reduction of the nitroolefins. To an ice-cooled suspension of lithium aluminum hydride (LiAlH₄) in tetrahydrofuran (THF) anh. is added dropwise sulfuric acid (H₂SO₄) 95-99% under an inert atmosphere and vigorous stirring. When hydrogen evolution has ceased the mixture is stirred for another 5-10 min. Next, a solution of the nitroolefin in THF anh. is added under ice-cooling at such a rate that the reaction becomes not too violent and the reaction temperature stays below 20-30° C. After completion of addition the mixture is brought to a gentle reflux for 3-5 min, and then again cooled with an ice-bath. Next, the mixture is cautiously quenched by successive and dropwise addition of anh. isopropanol (IPA) and then 2M sodium hydroxide solution (NaOH). Occasionally, THF is added to keep the mixture stirrable. When hydrolysis is complete, the mixture is filtered off and the filter cake is rinsed well with THF. The filtrate is concentrated in vacuo; purging the apparatus may be performed by applying an inert gas such as nitrogen or argon which prevents the formation of any unwanted carbamates.

General method for the hydrochloride salt formations. The base of the homoscaline or 3C-homoscaline is dissolved in approx. 30-50 times the mass of anh. diethyl ether containing 0.5% anh. IPA. The well stirred solution is cautiously neutralized by the addition of 2M anh. HCl in diethyl ether or 4M anh. HCl in dioxane and occasional cooling; the pH should not be far from neutral in order to not get a sticky mass during processing. The suspension obtained is filtered off, rinsed with diethyl ether, and dried in vacuo to get the final hydrochloride product.

### Examples—Preparation of the Aldehydes 2a-2v

4-Cyclobutoxy-3,5-dimethoxybenzaldehyde, 2a. According to the general method described, from 6.7 g syringaldehyde, 43 mL DMSO, 31 mg KI, 8.21 g K₂CO₃ and 5.0 g cyclobutyl bromide, 4.5 h reaction time, yield: 3.75 g (43.2%) brownish-beige solid. ¹H-NMR (CDCl₃): 1.46 (m, 1H, CH₂(CH₂)₂), 1.74 (m, 1H, CH₂(CH₂)₂), 2.26 (m, 4H, CH₂(CH₂)₂), 3.90 (s, 2 MeO), 4.70 (m, CHO—), 7.12 (s, 2 arom. H), 9.85 (s, CHO).

3,5-Dimethoxy-4-(1-methyl-allyloxy)benzaldehyde, 2b. According to the general method described, from 10.0 g syringaldehyde, 65 mL DMSO, 46 mg KI, 12.25 g K₂CO₃ and 5.1 g plus 2 g (2^{nd} addition after 2.5h) 3-chloro-1-butene, 4 h reaction time, yield: 4.66 g (35.9%) brownish oil. ¹H-NMR (CDCl₃): 1.44 (d, Me), 3.91 (s, 2 MeO), 4.83 (m, CHO—), 5.05 (m, 2H, H₂C═C), 5.93 (m, H₂C═CH), 7.12 (s, 2 arom. H), 9.87 (s, CHO).

4-But-3-enoxy-3,5-dimethoxy-benzaldehyde, 2c. According to the general method described, from 13.0 g syringaldehyde, 85 mL DMSO, 60 mg KI, 15.92 g K₂CO₃ and 9.8 g 4-chloro-1-butene, 2.5 h reaction time, yield: 9.76 g (57.9%) brownish oil. ¹H-NMR (CDCl₃): 2.53 (m, CH₂CH₂O), 3.92 (s, 2 MeO), 4.14 (t, CH₂O), 5.13 (m, 2H, H₂C═C), 5.91 (m, H₂C═CH), 7.14 (s, 2 arom. H), 9.86 (s, CHO).

3,5-Dimethoxy-4-(2,2,2-trifluoroethoxy)benzaldehyde, 2d. A mixture of 32.2 g (176.7 mmol) syringaldehyde and 87.3 g (266.1 mmol) Cs₂CO₃ in 320 mL DMSO anh. was stirred vigorously under N₂ for 2-3 min where after the flask was placed in an ice bath. Next, 49.5 g (30.7 mL; 213.3 mmol) 2,2,2-trifluoroethyl triflate were added during 2 min under vigorous stirring whereby the mixture quickly became better stirrable. After 15 min the mixture was poured into 1 L ice-water and then extracted with dichloromethane (DCM, 3×150 mL). The combined organic extracts were successively washed with NaOH 2M (2×100 mL) and water (3×200 mL), dried over Na₂SO₄ and concentrated in vacuo. There were obtained 41.05 g (87.9%) as a beige solid. ¹H-NMR (CDCl₃): 3.97 (s, 2×O—CH₃), 4.48 (q, ³J(H,F)=9 Hz, CH₂O), 7.17 (s, 2 arom. H), 9.91 (s, CHO).

3,5-Dimethoxy-4-(2-fluoroallyloxy)-benzaldehyde, 2e. According to the general method described, from 9.0 g syringaldehyde, 150 mL DMSO, 41 mg KI, 26.0 g Cs₂CO₃ and 5.1 g 3-chloro-2-fluoroprop-1-ene, 3 h reaction time, yield: 9.74 g (82.1%) 2e as a beige solid. ¹H-NMR (CDCl₃): 3.92 (s, 2 MeO), 4.64 (d, CH₂O—), 4.69 (dd and dd, superimposed, 2H, H₂C═C), 7.13 (s, 2 arom. H), 9.87 (s, CHO).

4-(2,2-Difluorovinyloxy)-3,5-dimethoxybenzaldehyde, 2f. A solution of 25.0 g (94.6 mmol) 3,5-dimethoxy-4-(2,2,2-trifluoroethoxy)benzaldehyde (2d) and 350 mg p-toluenesulfonic acid monohydrate in 75 mL MeOH anh. and 75 mL trimethyl orthoformate was held on reflux under nitrogen for 4 h. The mixture was cooled to r.t. and diluted with 600 mL diethyl ether and was washed with NaOH 2M (2×150 mL) and with brine (2×100 mL), dried over Na₂SO₄ and concentrated in vacuo (up to 70° C. in order to get rid of any hardly volatile impurities) to get 30.70 g (104.6%) of 3,5-dimethoxy-4-(2,2,2-trifluoroethoxy)benzaldehyde dimethyl acetal (7) as a clear yellowish liquid of a fruity odor. ¹H-NMR (CDCl₃; a complex spectrum was obtained): 3.32 and 3.62 (m, both belonging to CH(OMe)₂), 3.88 (s, 2 MeO), 4.34 (tq, CH₂O), 5.32 (m, CH), 6.71 (m, 2 arom. H). Next, BuLi 2.5M (23.20 mL, 3.0 eq) was added to a solution of 8.20 mL (5.87 g; 3.0 eq) diisopropylamine in THF at 0° C. This solution was added dropwise (10 min) to a solution of 6.00 g (19.34 mmol) 3,5-dimethoxy-4-(2,2,2-trifluoroethoxy)benzaldehyde dimethyl acetal (7) in 75 mL THF anh. at −78° C. under nitrogen. After 20 min a solution of 1.05 g (3 eq; 58.02 mmol) water in 30 mL THF anh. was added dropwise over a period of 10 min. Stirring at −78° C. was maintained for 1 h and then the reaction mixture was allowed to warm to 0° C. Next, the mixture was quenched by the dropwise addition of saturated NH₄Cl solution (40 mL) and then diluted with 400 mL diethyl ether. The layers were separated, and the org. layer was washed with NaHCO₃ sat. (2×150 mL), citric acid 5% (2×100 mL), water (2×100 mL), and finally with brine (1×100 mL), dried over Na₂SO₄ and concentrated in vacuo to get 5.36 g (95.5%) 4-(2,2-difluorovinyloxy)-3,5-dimethoxybenzaldehyde dimethyl acetal (8) as an orange oil. ¹H-NMR (CDCl₃; a complex spectrum was obtained): 3.34 and 3.62 (m, both belonging to CH(OMe)₂), 3.87 (s, 2 MeO), 5.36 (m, CH), 6.07 (dm, F₂CCH), 6.72 (m, 2 arom. H). Next, a mixture of 5.2 g (17.91 mmol) 4-(2,2-difluorovinyloxy)-3,5-dimethoxybenzaldehyde dimethyl acetal (8) and 50 mg pTsOH in THF-water (40 mL plus 80 mL) was heated under nitrogen to 85° C. After 2.5h the mixture was cooled to room temperature (RT), diluted with 150 mL DCM and washed once with saturated NaHCO₃ and water, dried over Na₂SO₄, filtered through a small amount of silica gel, the silica gel was further rinsed with DCM and the filtrate was concentrated in vacuo to get 4.21 g (96.2%) of 4-(2,2-difluorovinyloxy)-3,5-dimethoxybenzaldehyde (2f) as a white solid. ¹H-NMR (CDCl₃): 3.94 (s, 2 MeO), 6.18 (dd, (dd, ³J(H,F)=15.1 Hz and 3.1 Hz, F₂CCH), 7.16 (s, 2 arom. H), 9.90 (s, CHO).

4-(2,2-Difluoro-1-methyl-vinyloxy)-3,5-dimethoxybenzaldehyde, 2g. BuLi 2.5M (23.20 mL, 3.0 eq) was added to a solution of 8.20 mL (5.87 g; 3.0 eq) diisopropylamine in THF at 0° C. This solution was added dropwise (10 min) to a solution of 6.00 g (19.34 mmol) 3,5-dimethoxy-4-(2,2,2-trifluoroethoxy)benzaldehyde dimethyl acetal (7; preparation: see under 4-(2,2-difluorovinyloxy)-3,5-dimethoxybenzaldehyde, 2f) in 75 mL THF anh. at −78° C. under nitrogen. After 20 min, a solution of 3.61 mL (3 eq) methyl iodide in 30 mL THF anh. was added dropwise over a period of 10 min. Stirring at −78° C. was maintained for 1 h and then the reaction mixture was allowed to warm to 0° C. Next, the mixture was quenched by the dropwise addition of saturated NH₄Cl solution (40 mL) and then diluted with 400 mL diethyl ether. The layers were separated, and the org. layer was washed with NaHCO₃ sat. (2×150 mL), citric acid 5% (2×100 mL), water (2×100 mL) and finally with brine (1×100 mL), dried over Na₂SO₄ and concentrated in vacuo to get 5.53 g (94.0%) 4-(2,2-difluoro-1-methyl-vinyloxy)-3,5-dimethoxybenzaldehyde dimethyl acetal (9) as a brownish oil. ¹H-NMR (CDCl₃; a complex spectrum was obtained): 1.74 (t, Me), 3.34 and 3.61 (m, both belonging to CH(OMe)₂), 3.86 (s, 2 MeO), 5.36 (m, CH), 6.71 (m, 2 arom. H). Next, a mixture of 5.2 g (17.09 mmol) 4-(2,2-difluoro-1-methyl-vinyloxy)-3,5-dimethoxybenzaldehyde dimethyl acetal (9) and 50 mg pTsOH in THF-water (40 mL plus 80 mL) was heated under nitrogen to 85° C. After 2.5h, the mixture was cooled to RT, diluted with 150 mL DCM, and washed once with saturated NaHCO₃ and water, dried over Na₂SO₄, filtered through a small amount of silica gel, the silica gel was further rinsed with DCM and the filtrate was concentrated in vacuo to get 4.12 g (93.4%) of 4-(2,2-difluoro-1-methyl-vinyloxy)-3,5-dimethoxybenzaldehyde (2g) as an orangish oil. ¹H-NMR (CDCl₃): 1.81 (t, ⁴J(H,F)=4.3 Hz, MeC), 3.93 (s, 2 MeO), 7.15 (s, 2 arom. H), 9.89 (s, CHO).

4-(2,2-Difluoro-1-deuterovinyloxy)-3,5-dimethoxybenzaldehyde, 2h. In a similar procedure as described for compound 2f, 6.50 g (20.95 mmol) 3,5-dimethoxy-4-(2,2,2-trifluoro-ethoxy)benzaldehyde dimethyl acetal (7; preparation: see under 4-(2,2-difluorovinyloxy)-3,5-dimethoxybenzaldehyde, 2f) were treated with 3 eq in-situ-generated lithium diisopropylamide in THF anh. at −78° C. under nitrogen. After 20 min, 2.05 equivalents of a 2.5M solution of butyllithium in hexanes was added over a period of 10 min while keeping the temperature at −78° C. After another 20 min, a solution of excess deuterium oxide (2.16 g; 5.2 eq) in 30 mL THF anh. was added dropwise over a period of 10 min. Stirring at −78° C. was maintained for 1 h whereby the deuterium oxide progressively dissolved and reacted, and then the reaction mixture allowed to warm to 0° C. Workup was proceeded exactly as described for 2f to get 5.69 g (93.2%) 4-(2,2-difluoro-1-deuterovinyloxy)-3,5-dimethoxybenzaldehyde dimethyl acetal (10) as an orange oil. ¹H-NMR (CDCl₃; a complex spectrum was obtained): 3.34 and 3.62 (m, both belonging to CH(OMe)₂), 3.87 (s, 2 MeO), 5.36 (m, CH), 6.72 (m, 2 arom. H) The vinylic signal from the non-deuterated analog (at 6.07, dm, F₂CCH) was completely absent. ¹⁹F-NMR (CDCl₃): −98.9 and −99.4 (dm); −121.6 and −122.0 (dt). ESI+ data: [M+1]⁺=292.27; decomposes completely to the aldehyde; [M+1]⁺=246.2, found: 246.1. No traces of e/z=245 was found (no non-deutero analog). This acetal 10 was hydrolyzed exactly as described under the preparation of compound 2f to get 4.57 g (95.4%) of 4-(2,2-difluoro-1-deuterovinyloxy)-3,5-dimethoxybenzaldehyde (2h) as a white solid. ¹H-NMR (CDCl₃): 3.95 (s, 2 MeO), 7.14 (s, 2 arom. H), 9.90 (s, CHO). ¹⁹F-NMR (CDCl₃): −98.0 (d), −120.3 (dm).

4-(2,2-Difluoro-1-(trideuteromethyl)vinyloxy)-3,5-dimethoxybenzaldehyde, 2i. In a similar way as described for compound 2g, 6.00 g (19.34 mmol) 3,5-dimethoxy-4-(2,2,2-trifluoro-ethoxy)benzaldehyde dimethyl acetal (7; preparation: see under 4-(2,2-difluorovinyloxy)-3,5-dimethoxybenzaldehyde, 2f) were treated with 3 eq in-situ-generated lithium diisopropylamide in THF anh. at −78° C. under nitrogen. After 20 min a solution of 3.69 mL (3 eq) trideuteromethyl iodide in 30 mL THF anh. was added dropwise over a period of 10 min. Stirring at −78° C. was maintained for 1 h and then the reaction mixture was allowed to warm to 0° C. Workup was proceeded exactly as described for 2g to get 5.50 g (92.5%) 4-(2,2-difluoro-1-(trideuteromethyl)vinyloxy)-3,5-dimethoxybenzaldehyde dimethyl acetal (11) as an orange oil. ¹H-NMR (CDCl₃; a complex spectrum was obtained): 3.34 and 3.61 (m, both belonging to CH(OMe)₂), 3.86 (s, 2 MeO), 5.36 (m, CH), 6.71 (m, 2 arom. H). The vinylic methyl signal known from the non-deuterated analog (1.74, t, Me) was completely absent. ¹⁹F-NMR (CDCl₃): −103.7 and −104.0 (d); −119.3 and −119.6 (dt). ESI+ data: [M+1]⁺=308.31; decomposes completely to the aldehyde; [M+1]⁺=262.24, found: 262.1. No traces of e/z=261, 260 or 259 was found (no non-deutero analog). This acetal 11 was hydrolyzed exactly as described under the preparation of compound 2g to get 4.52 g (96.7%) of 4-(2,2-difluoro-1-(trideuteromethyl)vinyloxy)-3,5-dimethoxybenzaldehyde (2i) as an orange oil. ¹H-NMR (CDCl₃): ¹H-NMR (CDCl₃): 3.93 (s, 2 MeO), 7.14 (s, 2 arom. H), 9.89 (s, CHO). ¹⁹F-NMR (CDCl₃): −103.7 and −104.0 (d); −119.3 and −119.6 (dt).

3,5-Dimethoxy-4-(2-fluoroethoxy)-benzaldehyde, 2j. According to the general method described, from 10.94 g syringaldehyde, 70 mL DMSO, 50 mg KI, 13.4 g K₂CO₃ and 7.8 g 1-bromo-2-fluoroethane, 1 h reaction time. Yield: 11.3 g (83%) product as pale-yellow crystals. ¹H-NMR (CDCl₃): 3.93 (s, 2×MeO), 4.45 (dt, ³J(H,F)=36 Hz, CH₂O—), 4.84 (dt, ²J(H,F)=51 Hz, H₂FC), 7.18 (s, 2 arom. H), 9.92 (s, CHO).

4-(2,2-Difluoroethoxy)-3,5-dimethoxybenzaldehyde, 2k. According to the general method described, from 10.94 g syringaldehyde, 70 mL DMSO, 50 mg KI, 13.4 g K₂CO₃ and 8.7 g plus 1.5 g (2^{nd} addition after 0.5h) 1-bromo-2,2-difluoroethane, 1.5 h reaction time. Yield: 14.0 g (95%) product as a white solid. ¹H-NMR (CDCl₃): 3.96 (s, 2 MeO), 4.26 (dt, ³J(H,F)=12 Hz, CH₂O), 6.10 (tt, ²J(H,F)=54 Hz, CHF₂), 7.15 (s, 2 arom. H), 9.89 (s, CHO).

3,5-Dimethoxy-4-(3-fluoropropoxy)-benzaldehyde, 2l. According to the general method described, from 6.4 g syringaldehyde, 50 mL DMSO, 30 mg KI, 7.84 g K₂CO₃ and 5 g 1-bromo-3-fluoropropane, 1 h reaction time. Yield: 7.3 g (86%) product as an orange oil. ¹H-NMR (CDCl₃): 2.17 (dm, ³J(H,F)=24 Hz, CH₂CH₂O), 3.93 (s, 2 MeO); 4.23 (t, CH₂O), 4.77 (dt, ²J(H,F)=46 Hz, FCH₂), 7.15 (s, 2 arom. H), 9.90 (s, CHO).

3,5-Dimethoxy-4-(3-isobutoxy)-benzaldehyde, 2m. According to the general method described, from 5.47 g syringaldehyde, 40 mL DMSO, 30 mg KI, 6.7 g K₂CO₃ and 4.3 g plus 3.0 g plus 6.0 g (2^{nd} addition after 0.5h, 3^{rd} addition after 1 h) isobutyl bromide, 2 h reaction time. Yield: 6.6 g (92%) product as a bright orange oil. ¹H-NMR (CDCl₃): 1.05 (d, Me₂CH—), 2.10 (m, CH—CH₂—), 3.88 (d, —CH₂O—), 3.93 (s, 2 MeO), 7.13 (s, 2 arom. H), 9.88 (s, CHO).

3,5-Dimethoxy-4-propoxybenzaldehyde, 2n. According to the general method described, from 5.47 g syringaldehyde, 40 mL DMSO, 30 mg KI, 6.7 g K₂CO₃ and 4 g 1-bromopropane, 1 h reaction time. Yield: 6.25 g (93%) product as a bright orange oil. ¹H-NMR (CDCl₃): 1.04 (t, Me), 1.80 (m, MeCH₂—), 3.94 (s, 2 MeO), 4.09 (t, CH₂O—), 7.14 (s, 2 arom. H), 9.88 (s, CHO).

4-Allyloxy-3,5-dimethoxybenzaldehyde, 2o. According to the general method described, from 5.47 g syringaldehyde, 40 mL DMSO, 30 mg KI, 6.7 g K₂CO₃ and 2.5 g allyl chloride, 1 h reaction time. Yield: 5.83 g (87%) product as a beige solid. ¹H-NMR (CDCl₃): 3.94 (s, 2 MeO), 4.68 (d, CH₂O—), 5.23 (d, 1H, H₂C═C), 5.35 (d, 1H, H₂C═C), 6.10 (m, H₂C═CH), 7.16 (s, 2 arom. H), 9.90 (s, CHO).

3,5-Dimethoxy-4-isopropoxybenzaldehyde, 2p. According to the general method described, from 20 g syringaldehyde, 150 mL DMSO, 110 mg KI, 24.5 g K₂CO₃ and 18.5 g 2-bromopropane, 1 h reaction time. Yield: 24 g (97%) product as a bright-yellow oil. ¹H-NMR (CDCl₃): 1.32 (d, Me₂CH—), 3.92 (s, 2 MeO), 4.57 (m, Me₂CH), 7.12 (s, 2 arom. H), 9.87 (s, CHO).

3,5-Dimethoxy-4-methallyloxybenzaldehyde, 2q. According to the general method described, from 15 g syringaldehyde, 110 mL DMSO, 80 mg KI, 18.4 g K₂CO₃ and 8.0 g methallyl chloride, 1 h reaction time. Yield: 18.7 g (96%) product as an orange oil. ¹H-NMR (CDCl₃): 1.89 (s, MeC), 3.93 (s, 2 MeO), 4.56 (d, CH₂O—), 4.95 (s, 1H, H₂C═C), 5.09 (d, 1H, H₂C═C), 7.13 (s, 2 arom. H), 9.85 (s, CHO).

4-(1,3-Difluoroprop-2-yloxy)-3,5-dimethoxybenzaldehyde, 2r. According to the general method described, from 8.1 g syringaldehyde, 200 mL DMSO, 180 mg KI, 15.17 g K₂CO₃ and 7.9 g 1,3-difluoro-2-methanesulfonylpropane (prepared in analogy to DE3429048), 2 h reaction time, then 1 mL water was added and the temperature was increased to 100° C. for another 2 h, yield: 1.10 g (9.5%) product as a beige-brown solid. ¹H-NMR (CDCl₃): 3.96 (s, 2 MeO), 4.54 (m, (FCH₂)₂CH), 4.74 (dm, (FCH₂)₂CH), 7.16 (s, 2 arom. H), 9.91 (s, CHO).

3,5-Dimethoxy-4-(1,1,1-trifluoroprop-3-yloxy)-benzaldehyde, 2s. According to the general method described, from 10.0 g syringaldehyde, 200 mL DMSO, 200 mg KI, 15.17 g K₂CO₃ and 12.54 g plus 12.5 g (2^{nd} addition after 3 h) 1,1,1-trifluoropropyl iodide, 5 h reaction time, yield: 1.28 g (8.4%) product as an orangish oil. ¹H-NMR (CDCl₃): 2.67 (m, CF₃CH₂), 3.95 (s, 2 MeO), 4.30 (t, OCH₂), 7.15 (s, 2 arom. H), 9.90 (s, CHO).

3,5-Dimethoxy-4-vinyloxybenzaldehyde, 2t. The introduction of a vinyl ether function was adapted and modified from the protocol described by (McKinley & O'Shea, 2004). Cu(OAc)₂ (10.08 g, 54.88 mmol) in 370 mL DCM anh. was stirred for 10 min under air using a balloon. Next, 8.78 g (36.59 mmol) 2,4,6-trivinylcyclotriboroxane-pyridine complex, 10.0 g (54.9 mmol) syringaldehyde and 44.8 mL pyridine were added and the mixture was allowed to stir for 2 days; initially, after 8 h and after 24h there was air bubbled through the mixture for 1 min, each. The mixture was filtered through a silica gel pad. The filtrate was washed twice with NaOH 1M, water, twice with HCl 0.1M and brine. The org. layer was dried over MgSO₄ and concentrated in vacuo. There were obtained 5.80 g (51%) product as a beige solid. ¹H-NMR (CDCl₃): 3.90 (s, 2 MeO), 4.23 (dd, 1H, H₂C═C), 4.43 (dd, 1H, H₂C═C), 6.60 (dd, H₂C═CH), 7.14 (s, 2 arom. H), 9.90 (s, CHO).

4-Difluoromethoxy-3,5-dimethoxybenzaldehyde, 2u. The introduction of a 0-difluoromethyl substituent onto a phenol was adapted from (O'Shea et al., 2005). A mixture of 1.72 g (9.44 mmol) syringaldehyde, 2.88 g (18.88 mmol) sodium chlorodifluoroacetate and 1.57 g (11.3 mmol) K₂CO₃ in dimethylformamide (DMF) and H₂O (17 mL+2 mL) was degassed for 5 min with N₂. Then the mixture was heated to 100° C. (oil bath, preheated) for 4 h. The mixture was cooled to RT and 2.7 mL HCl 12M and 3.9 mL water were added. After stirring for 2 h 16 mL NaOH 2M were added and the mixture was diluted with Et₂O and water. The layers were separated, and the aqueous layer was further extracted with Et₂O (2×). The combined org. layers were washed with NaOH 2M (2×), water and brine, dried over Na₂SO₄ and concentrated in vacuo. Yield: 1.63 g (74%) product as a white solid. ¹H-NMR (CDCl₃): 3.95 (s, 2 MeO), 6.65 (t, ²J(H,F)=75.2 Hz, F₂CH), 7.13 (s, 2 arom. H), 9.92 (s, CHO).

3,5-Dimethoxy-4-trifluoromethoxybenzaldehyde, 2v. The introduction of a O-trifluoromethyl substituent onto a phenol was adapted from (Matsuya et al., 2007). To a solution of 5.83 g (32 mmol) syringaldehyde in dry 200 mL anh. DMF were added 5.40 g (38 mmol) K2CO3. The mixture was heated to −60° C., then 14.20 g (36 mmol) S-(trifluoromethyl)dibenzothiophenium trifluoromethanesulfonate were added portion wise at 50° C. (exothermic reaction), and the mixture was stirred for 2 h at RT and then for another 1 h at 75° C. The reaction mixture was diluted with water and extracted 3× with methyl-tert-butyl ether (MTBE). The combined organic layers were washed with NaOH 1 M (3×) and water (2×), dried over MgSO₄ and concentrated in vacuo. The crude residue (9.5 g) was purified by a short-path silica-gel column (DCM as eluate). Yield: 1.29 g (16%) product as a pale-yellowish solid. ¹H-NMR (CDCl₃): 3.98 (s, 2 MeO), 7.17 (s, 2 arom. H), 9.96 (s, CHO). A ¹⁹F-NMR spectrum (CDCl₃) showed a single peak at −57.9 ppm.

### Examples—Preparation of the Nitroolefines 3a-m; 3r-v and 4a-q; 4u

4-Cyclobutoxy-3,5-dimethoxy-β-nitrostyrene, 3a. According to the general method described, from 2.0 g 2a, 4.5 mL nitromethane, 100 μL butylamine, 100 μL acetic acid and 0.17 g molecular sieves, 25 min at 90° C. Yield: 1.92 g (81.2%) 3a as a yellow-orange solid. ¹H-NMR (CDCl₃): 1.47 (m, 1H, CH₂(CH₂)₂), 1.74 (m, 1H, CH₂(CH₂)₂), 2.24 (m, 4H, CH₂(CH₂)₂), 3.90 (s, 2 MeO), 4.65 (m, CHO—), 6.74 (s, 2 arom. H), 7.53 (d, CHNO₂), 7.93 (d, CH═CHNO₂).

1-(4-Cyclobutoxy-3,5-dimethoxyphenyl)-2-nitropropene, 4a. According to the general method described, from 1.75 g 2a, 4 mL nitroethane, 86 μL butylamine, 86 μL acetic acid and 0.15 g molecular sieves, 45 min at 90° C. Yield: 1.57 g (72.3%) 4a as a yellow-orange solid. 1H-NMR (CDCl3): 1.46 (m, 1H, CH2(CH2)2), 1.74 (m, 1H, CH2(CH2)2), 2.24 (m, 4H, CH2(CH2)2), 2.49 (d, MeC), 3.87 (s, 2 MeO), 4.63 (m, CHO—), 6.65 (s, 2 arom. H), 8.03 (s, CH═C).

3,5-Dimethoxy-4-(1-methylallyloxy)-β-nitrostyrene, 3b. According to the general method described, from 2.66 g 2b, 5.8 mL nitromethane, 130 μL butylamine, 130 μL acetic acid and 0.23 g molecular sieves, 25 min at 90° C. Yield: 2.62 g (83.3%) 3b as a yellow solid. 1H-NMR (CDCl3): 1.45 (d, Me), 3.87 (s, 2 MeO), 4.79 (m, CHO—), 5.04 (m, 2H, H2C═C), 5.95 (m, H2C═CH), 6.74 (s, 2 arom. H), 7.53 (d, CHNO2), 7.93 (d, CH═CHNO2).

1-(3,5-Dimethoxy-4-(1-methylallyloxy)phenyl)-2-nitropropene, 4b. According to the general method described, from 2.0 g 2b, 4.5 mL nitromethane, 100 μL butylamine, 100 μL acetic acid and 0.17 g molecular sieves, 45 min at 90° C. Yield: 1.87 g (75.3%) 4b as a yellow solid. ¹H-NMR (CDCl₃): 1.45 (d, Me), 2.49 (d, MeC), 3.88 (s, 2 MeO), 4.76 (m, CHO—), 5.06 (m, 2H, H₂C═C), 5.97 (m, H₂C═CH), 6.65 (s, 2 arom. H), 8.03 (s, CH═C).

4-But-3-enoxy-3,5-dimethoxy-β-nitrostyrene, 3c. According to the general method described, from 5.0 g 2c, 11 mL nitromethane, 250 μL butylamine, 250 μL acetic acid and 0.44 g molecular sieves, 45 min at 90° C. Yield: 5.00 g (84.6%) product as a yellow-orange solid. ¹H-NMR (CDCl₃): 2.53 (m, CH₂CH₂O), 3.89 (s, 2 MeO), 4.10 (t, CH₂O), 5.12 (m, 2H, H₂C═C), 5.91 (m, H₂C═CH), 6.75 (s, 2 arom. H), 7.53 (d, CHNO₂), 7.94 (d, CH═CHNO₂).

1-(4-But-3-enoxy-3,5-dimethoxyphenyl)-2-nitropropene, 4c. According to the general method described, from 4.75 g 2c, 11 mL nitroethane, 230 μL butylamine, 230 μL acetic acid and 0.42 g molecular sieves, 55 min at 90° C. Yield: 5.13 g (75.3%) product as an orange solid. ¹H-NMR (CDCl₃): 2.49 (d, MeC), 2.54 (m, CH₂CH₂O), 3.88 (s, 2 MeO), 4.08 (t, CH₂O), 5.13 (m, 2H, H₂C═C), 5.92 (m, H₂C═CH), 6.65 (s, 2 arom. H), 8.03 (s, CH═C).

3,5-Dimethoxy-4-(2,2,2-trifluoroethoxy)-β-nitrostyrene, 3d. According to the general method described, from 4.0 g 2d, 8 mL nitromethane, 180 μL butylamine, 180 μL acetic acid and 0.31 g molecular sieves, 20 min at 90° C. Yield: 3.52 g (75.7%) product as a bright yellow solid. ¹H-NMR (CDCl₃): 3.93 (s, 2×O—CH₃), 4.41 (q, ³J(H,F)=9 Hz, CH₂O), 6.78 (s, 2 arom. H), 7.53 (d, CHNO₂), 7.93 (d, CH═CHNO₂).

1-(3,5-Dimethoxy-4-(2,2,2-trifluoroethoxy)-phenyl)-2-nitropropene, 4d. According to the general method described, from 3.0 g 2d, 6 mL nitroethane, 130 μL butylamine, 130 μL acetic acid and 0.23 g molecular sieves, 55 min at 90° C. Yield: 2.89 g (79.2%) product as a bright yellow solid. ¹H-NMR (CDCl₃): 2.49 (d, MeC), 3.90 (s, 2×O—CH₃), 4.41 (q, ³J(H,F)=9 Hz, CH₂O), 6.68 (s, 2 arom. H), 8.02 (s, CH═C).

4-(2-Fluoroallyloxy)-3,5-dimethoxy-β-nitrostyrene, 3e. According to the general method described, from 5.0 g 2e, 11 mL nitromethane, 240 μL butylamine, 240 μL acetic acid and 0.43 g molecular sieves, 35 min at 90° C. Yield: 4.85 g (82.3%) product as a yellow solid. ¹H-NMR (CDCl₃): 3.90 (s, 2 MeO), 4.61 (d, ³J(H,F)=14 Hz, CH₂O—), 4.69 (dm, ³J(H,F)=˜68 Hz (partially superimposed), 1H, H₂C═C), 4.74 (m, 1H, H₂C═C), 6.76 (s, 2 arom. H), 7.53 (d, CHNO₂), 7.93 (d, CH═CHNO₂).

1-(4-(2-Fluoroallyloxy)-3,5-dimethoxyphenyl)-2-nitropropene, 4e. According to the general method described, from 4.7 g 2e, 10 mL nitroethane, 230 μL butylamine, 230 μL acetic acid and 0.41 g molecular sieves, 65 min at 90° C. Yield: 5.09 g (87.5%) product as a yellow solid. ¹H-NMR (CDCl₃): 2.49 (d, MeC), 3.88 (s, 2 MeO), 4.59 (d, ³J(H,F)=14 Hz, CH₂O—), 4.70 (dd, ³J(H,F)=64 Hz, ²J=3.9 Hz, 1H, H₂C═C), 4.75 (d, ²J=3.9 Hz, 1H, H₂C═C), 6.66 (s, 2 arom. H), 8.03 (s, CH═C).

4-(2,2-Difluorovinyloxy)-3,5-dimethoxy-β-nitrostyrene, 3f. According to the general method described, from 2.0 g 2f, 4.2 mL nitromethane, 95 μL butylamine, 95 μL acetic acid and 0.30 g molecular sieves, 70 min at 70° C. Yield: 2.01 g (85.5%) product as a bright yellow solid. ¹H-NMR (CDCl₃): 3.91 (s, 2 MeO), 6.16 (dd, ³J(H,F)=15.2 Hz and 3.1 Hz, F₂CCH), 6.76 (s, 2 arom. H), 7.54 (d, CHNO₂), 7.93 (d, CH═CHNO₂).

1-(4-(2,2-Difluorovinyloxy)-3,5-dimethoxyphenyl)-2-nitropropene, 4f. According to the general method described, from 2.0 g 2f, 4 mL nitroethane, 90 μL butylamine, 90 μL acetic acid and 0.30 g molecular sieves, 85 min at 80° C. Yield: 2.23 g (97.8%) product as a bright yellow solid. ¹H-NMR (CDCl₃): 2.47 (d, MeC), 3.89 (s, 2 MeO), 6.15 (dd, ³J(H,F)=15.3 Hz and 2.9 Hz, F₂CCH), 6.64 (s, 2 arom. H), 8.02 (s, CH═C).

4-(2,2-Difluoro-1-methyl-vinyloxy)-3,5-dimethoxy-β-nitrostyrene, 3g. According to the general method described, from 2.0 g 2g, 4.2 mL nitromethane, 95 μL butylamine, 95 μL acetic acid and 0.30 g molecular sieves, 50 min at 70° C. Yield: 1.72 g (73.3%) product as spectacular orange-golden glistening plates. ¹H-NMR (CDCl₃): 1.80 (t, ⁴J(H,F)=4.2 Hz, MeC), 3.90 (s, 2 MeO), 6.76 (s, 2 arom. H), 7.54 (d, CHNO₂), 7.94 (d, CH═CHNO₂).

1-(4-(2,2-Difluoro-1-methyl-vinyloxy)-3,5-dimethoxyphenyl)-2-nitropropene, 4g. According to the general method described, from 2.0 g 2g, 4 mL nitroethane, 90 μL butylamine, 90 μL acetic acid and 0.30 g molecular sieves, 65 min at 80° C. Yield: 1.50 g (61.4%) product as a pale-yellow solid. ¹H-NMR (CDCl₃): 1.79 (t, ⁴J(H,F)=4.2 Hz, MeC), 2.48 (d, MeCNO₂), 3.87 (s, 2 MeO), 6.65 (s, 2 arom. H), 8.02 (s, CH═C).

4-(2,2-Difluoro-1-deuterovinyloxy)-3,5-dimethoxy-β-nitrostyrene, 3h. According to the general method described, from 2.56 g 2h, 5.5 mL nitromethane, 120 μL butylamine, 120 μL acetic acid and 0.22 g molecular sieves, 65 min at 70° C. Yield: 2.05 g (68.1%) product as bright-yellow crystals. ¹H-NMR (CDCl₃): 3.91 (s, 2 MeO), 6.76 (s, 2 arom. H), 7.54 (d, CHNO₂), 7.93 (d, CH═CHNO₂).

1-(4-(2,2-Difluoro-1-deuterovinyloxy)-3,5-dimethoxyphenyl)-2-nitropropene, 4h. According to the general method described, from 2.0 g 2h, 4.5 mL nitroethane, 95 μL butylamine, 95 μL acetic acid and 0.18 g molecular sieves, 80 min at 80° C. Yield: 1.72 g (69.8%) product as bright-yellow crystals. ¹H-NMR (CDCl₃): 2.47 (d, MeC), 3.89 (s, 2 MeO), 6.65 (s, 2 arom. H), 8.02 (s, CH═C).

4-(2,2-Difluoro-1-(trideuteromethyl)vinyloxy)-3,5-dimethoxy-β-nitrostyrene, 3i. According to the general method described, from 2.50 g 2i, 5 mL nitromethane, 110 μL butylamine, 110 μL acetic acid and 0.20 g molecular sieves, 55 min at 70° C. Yield: 1.90 g (65.3%) product as yellow crystals. ¹H-NMR (CDCl₃): 3.90 (s, 2 MeO), 6.76 (s, 2 arom. H), 7.54 (d, CHNO₂), 7.94 (d, CH═CHNO₂).

1-(4-(2,2-Difluoro-1-(trideuteromethyl)vinyloxy)-3,5-dimethoxyphenyl)-2-nitropropene, 4i. According to the general method described, from 2.0 g 2i, 4 mL nitroethane, 90 μL butylamine, 90 μL acetic acid and 0.16 g molecular sieves, 80 min at 80° C. Yield: 1.25 g (51.3%) product as yellowish crystals. ¹H-NMR (CDCl₃): 2.48 (d, MeCNO₂), 3.87 (s, 2 MeO), 6.65 (s, 2 arom. H), 8.03 (s, CH═C).

3,5-Dimethoxy-4-(2-fluoroethoxy)-β-nitrostyrene, 3j. According to the general method described, from 6.0 g 2j, 15 mL nitromethane, 200 μL butylamine and 200 μL acetic acid, 30 min at reflux (oil bath 110° C.). Yield: 4.23 g (59%) product as a brownish-yellow solid. ¹H-NMR (CDCl₃): 3.90 (s, 2 MeO), 4.33 (dt, ³J(H,F)=29 Hz, CH₂O), 4.72 (dt, ²J(H,F)=52 Hz, CH₂F), 6.76 (s, 2 arom. H), 7.56 (d, CHNO₂), 7.94 (d, CH═CHNO₂).

1-(3,5-Dimethoxy-4-(2-fluoroethoxy)-phenyl)-2-nitropropene, 4j. According to the general method described, from 5.3 g 2j, 8 mL nitroethane, 200 μL butylamine and 200 μL acetic acid, 75 min at reflux (oil bath 120° C.); during the last 15 min the water formed was removed azeotropically. Yield: 5.34 g (81%) product as a bright yellow solid. ¹H-NMR (CDCl₃): 2.51 (s, MeC), 3.91 (s, 2 MeO), 4.33 (dt, ³J(H,F)=29 Hz, CH₂O), 4.72 (dt, ²J(H,F)=48 Hz, CH₂F), 6.68 (s, 2 arom. H), 8.05 (s, CH═C).

4-(2,2-Difluoroethoxy)-3,5-dimethoxy-β-nitrostyrene, 3k. According to the general method described, from 7.0 g 2k, 20 mL nitromethane, 95 μL butylamine and 95 μL acetic acid, 20 min at reflux (oil bath 110° C.). Yield: 5.22 g (64%) product as a bright yellow solid. A mixture of E- and Z-isomer was obtained. ¹H-NMR (CDCl₃): (E)-Isomer: 3.93 (s, 2 MeO), 4.25 (dt, ³J(H,F)=13 Hz, CH₂O), 6.11 (tt, ²J(H,F)=55 Hz, CHF₂), 6.78 (s, 2 arom. H), 7.54 (d, ³J=14 Hz, CHNO₂), 7.93 (d, ³J=14 Hz, CH═CHNO₂). (Z)-Isomer: 3.69 (s, 2 MeO), 4.10 (dt, ³J(H,F)=13 Hz, CH₂O), 4.82 (d, ³J=5 Hz, CHNO₂), 5.66 (d, ³J=5 Hz, CH═CHNO₂), 6.04 (tt, ²J(H,F)=55 Hz, CHF₂), 6.12 (s, 2 arom. H). EI-MS: 290 (15, [M+1]⁺), 289 (100, M⁺), 224 (23, [M-65]⁺), 177 (96, [M-112]⁺).

1-(4-(2,2-Difluoroethoxy)-3,5-dimethoxyphenyl)-2-nitropropene, 4k. According to the general method described, from 7.0 g 2k, 15 mL nitroethane, 300 μL butylamine and 300 μL acetic acid, 50 min at reflux (oil bath 120° C.); during the last 15 min the water formed was removed azeotropically. Yield: 5.5 g (64%) product as a yellow solid. ¹H-NMR (CDCl₃): 2.50 (s, MeC), 3.91 (s, 2 MeO), 4.23 (dt, ³J(H,F)=13 Hz, CH₂O), 6.12 (tt, ²J(H,F)=55 Hz, CHF₂), 6.67 (s, 2 arom. H), 8.04 (s, CH═C).

3,5-Dimethoxy-4-(3-fluoropropoxy)-β-nitrostyrene, 3l. According to the general method described, from 4.0 g 21, 10 mL nitromethane, 200 μL butylamine and 200 μL acetic acid, 25 min at reflux (oil bath 110° C.). Yield: 2.37 g (50%) product as a yellow solid. ¹H-NMR (CDCl₃): 2.15 (dm, ³J(H,F)=26 Hz, CH₂CH₂O), 3.91 (s, 2 MeO), 4.19 (t, CH₂O), 4.73 (dt, ²J(H,F)=47 Hz, FCH₂), 6.77 (s, 2 arom. H), 7.55 (d, CHNO₂), 7.95 (d, CH═CHNO₂).

1-(3,5-Dimethoxy-4-(3-fluoropropoxy)-phenyl)-2-nitropropene, 4l. According to the general method described, from 3.3 g 21, 8 mL nitroethane, 100 μL butylamine and 100 μL acetic acid, 40 min at reflux (oil bath 120° C.); during the last 15 min the water formed was removed azeotropically. Yield: 3.31 g (81%) product as a yellow solid. ¹H-NMR (CDCl₃): 2.16 (dm, ³J(H,F)=26 Hz, CH₂CH₂O), 2.51 (s, MeC), 3.89 (s, 2 MeO), 4.17 (t, CH₂O), 4.74 (dt, ²J(H,F)=47 Hz, FCH₂), 6.67 (s, 2 arom. H), 8.05 (s, CH═C).

3,5-Dimethoxy-4-(3-isobutoxy)-β-nitrostyrene, 3m. According to the general method described, from 3.3 g 2m, 8 mL nitromethane, 150 μL butylamine and 150 μL acetic acid, 30 min at reflux (oil bath 110° C.). Yield: 2.14 g (55%) product as a yellow solid. ¹H-NMR (CDCl₃): 1.04 (d, Me₂CH—), 2.08 (m, CH—CH₂—), 3.82 (d, —CH₂O—), 3.90 (s, 2 MeO), 6.77 (s, 2 arom. H), 7.55 (d, CHNO₂), 7.95 (d, CH═CHNO₂).

1-(3,5-Dimethoxy-4-(3-isobutoxy)phenyl)-2-nitropropene, 4m. According to the general method described, from 3.3 g 2m, 8 mL nitroethane, 150 μL butylamine and 90 μL acetic acid, 60 min at reflux (oil bath 120° C.); during the last 15 min the water formed was removed azeotropically. The reaction mixture was concentrated in vacuo, dissolved in DCM and washed 3× with water, dried over MgSO₄ and again concentrated in vacuo. Yield: 4.05 g (99%) product as an orange oil. ¹H-NMR (CDCl₃): 1.04 (d, Me₂CH—), 2.08 (m, CH—CH₂—), 2.51 (s, MeCH); 3.81 (d, —CH₂O—), 3.88 (s, 2 MeO), 6.67 (s, 2 arom. H), 8.06 (s, CH═C).

1-(3,5-Dimethoxy-4-propoxyphenyl)-2-nitropropene, 4n. According to the general method described, from 6.25 g 2n, 13 mL nitroethane, 250 μL butylamine and 250 μL acetic acid, 30 min at reflux (oil bath 120° C.); during the last 15 min the water formed was removed azeotropically. Yield: 4.7 g (60%) product as a yellow solid. ¹H-NMR (CDCl₃): 1.04 (t, MeCH₂), 1.80 (m, MeCH₂—), 2.51 (s, MeC), 3.89 (s, 2 MeO), 4.01 (t, CH₂O—), 6.68 (s, 2 arom. H), 8.06 (s, CH═C).

1-(4-Allyloxy-3,5-dimethoxyphenyl)-2-nitropropene, 4o. According to the general method described, from 5.8 g 2o, 12 mL nitroethane, 250 μL butylamine and 250 μL acetic acid, 60 min at reflux (oil bath 120° C.); during the last 15 min the water formed was removed azeotropically. Yield: 5.13 g (74%) product as a bright yellow solid. ¹H-NMR (CDCl₃): 2.50 (s, MeC), 3.90 (s, 2 MeO), 4.59 (d, CH₂O—), (d, 1H, H₂C═C), 5.34 (d, 1H, H₂C═C), 6.15 (m, H₂C═CH), 6.67 (s, 2 arom. H), 8.05 (s, CH═C).

1-(3,5-Dimethoxy-4-isopropoxyphenyl)-2-nitropropene, 4p. According to the general method described, from 6g 2p, 12 mL nitroethane, 270 μL butylamine and 270 μL acetic acid, 55 min at reflux (oil bath 120° C.); during the last 15 min the water formed was removed azeotropically. Yield: 4.75 g (63%) product as a yellow solid. ¹H-NMR (CDCl₃): 1.33 (d, Me₂CH—), 2.51 (s, MeC), 3.88 (s, 2 MeO), 4.47 (m, Me₂CH), 6.68 (s, 2 arom. H), 8.06 (s, CH═C).

1-(3,5-Dimethoxy-4-methallyloxyphenyl)-2-nitropropene, 4q. According to the general method described, from 3.7 g 2q, 8 mL nitroethane, 160 μL butylamine and 160 μL acetic acid, 40 min at reflux (oil bath 120° C.); during the last 15 min the water formed was removed azeotropically. Yield: 3.2 g (70%) product as an orange solid. ¹H-NMR (CDCl₃): 1.90 (s, MeC═), 2.51 (s, MeC), 3.89 (s, 2 MeO), 4.50 (s, CH₂O), 4.95 (s, 1H, H₂C═C), 5.08 (d, 1H, H₂C═C), 6.68 (s, 2 arom. H), 8.05 (s, CH═C).

4-(1,3-Difluoroprop-2-yloxy)-3,5-dimethoxy-β-nitrostyrene, 3r. According to the general method described, from 0.65 g 2r, 3 mL nitromethane, 50 μL butylamine and 50 μL acetic acid, 25 min at 70° C. Yield: 0.61 g (81%) product as a yellow solid. ¹H-NMR (CDCl₃): 3.93 (s, 2 MeO), 4.50 (m, (FCH₂)₂CH), 4.73 (dm, (FCH₂)₂CH), 6.79 (s, 2 arom. H), 7.56 (d, CHNO₂), 7.96 (d, CH═CHNO₂).

3,5-Dimethoxy-4-(1,1,1-trifluoroprop-3-yloxy)-β-nitrostyrene, 3s. According to the general method described, from 1.26 g 2s, 3 mL nitromethane, 80 μL butylamine and 80 μL acetic acid, 15 min at 95° C. Yield: 1.10 g (76%) product as an orange solid. ¹H-NMR (CDCl₃): 2.66 (m, CF₃CH₂), 3.92 (s, 2 MeO), 4.27 (t, OCH₂), 6.78 (s, 2 arom. H), 7.56 (d, CHNO₂), 7.96 (d, CH═CHNO₂).

3,5-Dimethoxy-4-vinyloxy-β-nitrostyrene, 3t. According to the general method described, from 3.5 g 2t, 10 mL nitromethane, 150 μL butylamine, 150 μL acetic acid and 3.0 g molecular sieves, 25 min at 95° C. Yield: 3.67 g (87.0%) product as a bright yellow solid. ¹H-NMR (CDCl₃): 3.92 (s, 2 MeO), 4.27 (dd, 1H, H₂C═C), 4.43 (dd, 1H, H₂C═C), 6.61 (dd, H₂C═CH), 6.81 (s, 2 arom. H), 7.57 (d, CHNO₂), 7.97 (d, CH═CHNO₂).

4-Difluoromethoxy-3,5-dimethoxy-β-nitrostyrene, 3u. According to the general method described, from 1.0 g 2u, 3 mL nitromethane, 30 μL butylamine and 30 μL acetic acid, 40 min at 95° C. Yield: 0.95 g (80%) product as a soft-yellowish solid. ¹H-NMR (CDCl₃): 3.92 (s, 2 MeO), 6.61 (t, ²J(H,F)=76 Hz, F₂CH), 6.77 (s, 2 arom. H), 7.54 (d, CHNO₂), 7.93 (d, CH═CHNO₂).

1-(4-Difluoromethoxy-3,5-dimethoxyphenyl)-2-nitropropene, 4u. According to the general method described, from 0.80 g 2u, 1 mL nitroethane, 20 μL butylamine and 20 μL acetic acid, 90 min at 95° C. Yield: 0.87 g (87%) product as a soft-yellow solid. ¹H-NMR (CDCl₃): 2.46 (s, MeC), 3.90 (s, 2 MeO), 6.60 (t, ²J(H,F)=75.9 Hz, F₂CH), 6.65 (s, 2 arom. H), 8.01 (s, CH═C).

3,5-Dimethoxy-4-trifluoromethoxy-β-nitrostyrene, 3v. According to the general method described, from 1.27 g 2v, 3 mL nitromethane, 60 μL butylamine, 60 μL acetic acid and 0.1 g molecular sieves, 30 min at 95° C. Yield: 1.31 g (88%) product as a pale-yellow solid. ¹H-NMR (CDCl₃): 3.94 (s, 2 MeO), 6.79 (s, 2 arom. H), 7.57 (d, CHNO₂), 7.96 (d, CH═CHNO₂).

### Examples—Alane-Promoted Reduction of the Nitroolefines to the Amines and Conversion to their Salts: Preparation of the Homo-Scales and 3C-Homoscalines 5a-m; 5r-v and 6a-q; 6u

4-Cyclobutoxy-3,5-dimethoxyphenethylamine hydrochloride (CB; Cyclobuscaline), 5a. According to the general method described, from 1.90 g 3a, 0.96 g LiAlH₄, 0.67 mL H₂SO₄, 21 mL plus 15 mL THF, 4.0 mL IPA and 3.1 mL NaOH 2M. Hydrochloride salt formation according to the general method described. Yield: 1.32 g (67.4%) product as a white solid. ¹H-NMR (D₂O): 1.21 (m, 1H, CH₂(CH₂)₂), 1.64 (m, 1H, CH₂(CH₂)₂), 1.93 (m, 4H, CH₂(CH₂)₂), 2.73 (t, ArCH₂), 3.05 (t, CH₂NH₃⁺), 3.62 (s, 2 MeO), 4.31 (m, CHO—), 6.46 (s, 2 arom. H).

4-Cyclobutoxy-3,5-dimethoxyamphetamine hydrochloride (3C-CB), 6a. According to the general method described, from 1.55 g 4a, 0.75 g LiAlH₄, 0.52 mL H₂SO₄, 16 mL plus 12 mL THF, 3.1 mL IPA and 2.4 mL NaOH 2M. Hydrochloride salt formation according to the general method described. Yield: 1.48 g (92.8%) product as a white solid. ¹H-NMR (D₂O): 1.31 (d, MeCH), 1.43 (m, 1H, CH₂(CH₂)₂), 1.68 (m, 1H, CH₂(CH₂)₂), 2.16 (m, 4H, CH₂(CH₂)₂), 2.90 (d, ArCH₂), 3.64 (m, CHNH₃⁺), 3.85 (s, 2 MeO), 4.55 (m, CHO—), 6.67 (s, 2 arom. H).

3,5-Dimethoxy-4-(1-methylallyloxy)phenethylamine hydrochloride (MAL-2; 1-Methallylscaline), 5b. According to the general method described, from 2.60 g 3b, 1.32 g LiAlH₄, 0.92 mL H₂SO₄, 30 mL plus 15 mL THF, 5.5 mL IPA and 4.2 mL NaOH 2M. Hydrochloride salt formation according to the general method described. Yield: 1.81 g (67.5%) product as a white solid. ¹H-NMR (DMSO-d₆): 1.27 (d, Me), 2.83 (t, ArCH₂), 3.03 (m, CH₂NH₃⁺), 3.76 (s, 2 MeO), 4.57 (m, CHO—), 5.03 (m, 2H, H₂C═C), 5.89 (m, H₂C═CH), 6.55 (s, 2 arom. H), 8.12 (bs, CH₂NH₃⁺).

3,5-Dimethoxy-4-(1-methylallyloxy)amphetamine hydrochloride (3C-MAL-2), 6b. According to the general method described, from 1.85 g 4b, 0.89 g LiAlH₄, 0.62 mL H₂SO₄, 20 mL plus 12 mL THF, 3.7 mL IPA and 2.9 mL NaOH 2M. Hydrochloride salt formation according to the general method described. Yield: 1.21 g (81.2%) product as a white solid. ¹H-NMR (D₂O): 1.23 (d, MeCH), 1.31 (d, MeCHO), 2.80 (d, ArCH₂), 3.57 (m, CHNH₃⁺), 3.76 (s, 2 MeO), 4.67 (m, CHO), 5.00 (dm, 2H, H₂C═C), 5.87 (m, H₂C═CH), 6.57 (s, 2 arom. H).

4-But-3-enoxy-3,5-dimethoxyphenethylamine hydrochloride (BE; Butenylscaline), 5c. According to the general method described, from 4.98 g 3c, 2.52 g LiAlH₄, 1.76 mL H₂SO₄, 55 mL plus 20 mL THF, 10.5 mL IPA and 8.0 mL NaOH 2M. Hydrochloride saltformation according to the general method described. Yield: 3.41 g (74.7%) product as a white solid. ¹H-NMR (D₂O): 2.38 (m, CH₂CH₂O), 2.87 (t, ArCH₂), 3.19 (t, CH₂NH₃⁺), 3.77 (s, 2 MeO), 3.92 (t, CH₂O), 5.07 (m, 2H, H₂C═C), 5.83 (m, H₂C═CH), 6.60 (s, 2 arom. H).

4-But-3-enoxy-3,5-dimethoxyamphetamine hydrochloride (3C-BE), 6c. According to the general method described, from 2.60 g 4c, 1.32 g LiAlH₄, 0.92 mL H₂SO₄, 30 mL plus 15 mL THF, 5.5 mL IPA and 4.2 mL NaOH 2M. Hydrochloride salt formation according to the general method described. Yield: 3.76 g (88.5%) product as a white solid. ¹H-NMR (D₂O): 1.22 (d, MeCH), 2.39 (m, CH₂CH₂O), 2.81 (d, ArCH₂), 3.55 (m, CHNH₃⁺), 3.77 (s, 2 MeO), 3.93 (t, CH₂O), 5.07 (m, 2H, H₂C═C), 5.85 (m, H₂C═CH), 6.57 (s, 2 arom. H).

3,5-Dimethoxy-4-(2,2,2-trifluoroethoxy)phenethylamine hydrochloride (TFE; Trifluoroescaline), 5d. According to the general method described, from 3.5 g 3d, 1.61 g LiAlH₄, 1.13 mL H₂SO₄, 35 mL plus 15 mL THF, 6.7 mL IPA and 5.1 mL NaOH 2M. Hydrochloride salt formation according to the general method described. Yield: 2.52 g (70%) product as a white solid. ¹H-NMR (D₂O): 2.98 (t, ArCH₂), 3.29 (t, CH₂NH₃⁺), 3.89 (s, 2 MeO), 4.49 (q, ³J(H,F)=9 Hz, CH₂O), 6.72 (s, 2 arom. H).

3,5-Dimethoxy-4-(2,2,2-trifluoroethoxy)amphetamine hydrochloride (3C-TFE), 6d. According to the general method described, from 2.87 g 4d, 1.26 g LiAlH₄, 0.88 mL H₂SO₄, 30 mL plus 15 mL THF, 5.3 mL IPA and 4.0 mL NaOH 2M. Hydrochloride salt formation according to the general method described. Yield: 2.41 g (81.8%) product as a white solid. ¹H-NMR (D₂O): 1.29 (d, MeCH), 2.89 (d, ArCH₂), 3.63 (m, CHNH₃⁺), 3.86 (s, 2 MeO), 4.45 (q, ³J(H,F)=9 Hz, CH₂O), 6.66 (s, 2 arom. H).

4-(2-Fluoroallyloxy)-3,5-dimethoxyphenethylamine hydrochloride (FAL; Fluoroallylscaline), 5e. According to the general method described, from 4.41 g 3e, 2.20 g LiAlH₄, 1.54 mL H₂SO₄, 50 mL plus 20 mL THF, 9.2 mL IPA and 7.0 mL NaOH 2M. Hydrochloride salt formation according to the general method described. Yield: 3.20 g (70.4%) product as a white solid. ¹H-NMR (DMSO-d₆): 2.84 (t, ArCH₂), 3.04 (m, CH₂NH₃⁺), 3.78 (s, 2 MeO), 4.41 (d, ³J(H,F)=15 Hz, CH₂O—), 4.71 (dd, ³J(H,F)=30 Hz, ²J(H,H)=3.1 Hz, 1H, H₂C═C), 4.82 (m, 1H, H₂C═C), 6.59 (s, 2 arom. H), 8.12 (bs, CH₂NH₃⁺). ¹⁹F-NMR (DMSO-d₆): −104.0 (s).

4-(2-Fluoroallyloxy)-3,5-dimethoxamphetamine hydrochloride (3C-FAL), 6e. According to the general method described, from 4.65 g 4e, 2.22 g LiAlH₄, 1.55 mL H₂SO₄, 50 mL plus 20 mL THF, 9.2 mL IPA and 7.1 mL NaOH 2M. Hydrochloride salt formation according to the general method described. Yield: 3.82 g (79.7%) product as a white solid. ¹H-NMR (DMSO-d₆): 1.16 (d, MeCH), 2.65 (m, 1H, ArCH₂), 2.96 (m, 1H, ArCH₂), 3.43 (m, CHNH₃⁺), 3.78 (s, 2 MeO), 4.42 (d, ³J(H,F)=16 Hz, CH₂O), 4.71 (dd, ³J(H,F)=30 Hz, ²J(H,H)=3.1 Hz, 1H, H₂C═C), 4.82 (m, 1H, H₂C═C), 6.58 (s, 2 arom. H), 8.20 (bs, CH₂NH₃⁺). ¹⁹F-NMR (DMSO-d₆): −104.0 (s).

4-(2,2-Difluorovinyloxy)-3,5-dimethoxyphenethylamine hydrochloride (DFV; Difluoroviscaline), 5f. According to the general method described, from 2.01 g 3f, 0.99 g LiAlH₄, 0.69 mL H₂SO₄, 22 mL plus 10 mL THF, 4.1 mL IPA and 3.2 mL NaOH 2M. Hydrochloride salt formation according to the general method described. Yield: 1.34 g (64.7%) product as a white solid. ¹H-NMR (D₂O): 2.88 (t, ArCH₂), 3.20 (t, CH₂NH₃⁺), 3.79 (s, 2 MeO), 6.16 (dd, ³J(H,F)=15.9 Hz and 2.7 Hz, F₂CCH), 6.63 (s, 2 arom. H). ¹⁹F-NMR (D₂O): −99.6 (d), −121.2 (d).

4-(2,2-Difluorovinyloxy)-3,5-dimethoxyamphetamine hydrochloride (3C-DFV), 6f. According to the general method described, from 2.23 g 4f, 1.05 g LiAlH₄, 0.73 mL H₂SO₄, 25 mL plus 10 mL THF, 4.4 mL IPA and 3.3 mL NaOH 2M. Hydrochloride salt formation according to the general method described. Yield: 1.82 g (79.4%) product as a white solid. ¹H-NMR (D₂O): 1.22 (d, MeCH), 2.81 (d, ArCH₂), 3.56 (m, CHNH₃⁺), 3.79 (s, 2 MeO), 6.16 (dd, ³J(H,F)=15.9 Hz and 3.0 Hz, F₂CCH), 6.60 (s, 2 arom. H). ¹⁹F-NMR (D₂O): −99.6 (d), −121.2 (d).

4-(2,2-Difluoro-1-methyl-vinyloxy)-3,5-dimethoxyphenethylamine hydrochloride (DFIPRE; Difluoroisopropenylscaline), 5g. According to the general method described, from 1.72 g 3f, 0.81 g LiAlH₄, 0.56 mL H₂SO₄, 20 mL plus 10 mL THF, 3.4 mL IPA and 2.6 mL NaOH 2M. Hydrochloride salt formation according to the general method described. Yield: 1.26 g (71.2%) product as a white solid. ¹H-NMR (D₂O): 1.69 (t, ⁴J(H,F)=4.5 Hz, MeC), 2.98 (t, ArCH₂), 3.29 (t, CH₂NH₃⁺), 3.87 (s, 2 MeO), 6.73 (s, 2 arom. H). ¹⁹F-NMR (D₂O): −104.5 (d), −119.1 (d).

4-(2,2-Difluoro-1-methyl-vinyloxy)-3,5-dimethoxyamphetamine hydrochloride (3C-DFIPRE), 6g. According to the general method described, from 1.50 g 4f, 0.67 g LiAlH₄, 0.47 mL H₂SO₄, 20 mL plus 10 mL THF, 2.8 mL IPA and 2.1 mL NaOH 2M. Hydrochloride salt formation according to the general method described. Yield: 0.99 g (64.4%) product as a white solid. ¹H-NMR (D₂O): 1.22 (d, MeCH), 1.61 (t, ⁴J(H,F)=4.5 Hz, MeCO), 2.83 (d, ArCH₂), 3.56 (m, CHNH₃⁺), 3.78 (s, 2 MeO), 6.62 (s, 2 arom. H). ¹⁹F-NMR (D₂O): −104.5 (d), −119.1 (d).

4-(2,2-Difluoro-1-deuterovinyloxy)-3,5-dimethoxyphenethylamine hydrochloride (Deutero-DFV; Deuterodifluoroviscaline), 5h. According to the general method described, from 2.04 g 3h, 1.00 g LiAlH₄, 0.70 mL H₂SO₄, 22 mL plus 10 mL THF, 4.2 mL IPA and 3.2 mL NaOH 2M. Hydrochloride salt formation according to the general method described. Yield: 0.82 g (38.8%) product as a white solid. ¹H-NMR (D₂O): 2.88 (t, ArCH₂), 3.19 (t, CH₂NH₃⁺), 3.78 (s, 2 MeO), 6.62 (s, 2 arom. H). ¹⁹F-NMR (D₂O): −99.8 (d), −121.3 (d).

4-(2,2-Difluoro-1-deuterovinyloxy)-3,5-dimethoxymphetamine hydrochloride (Deutero-3C-DFV), 6h. According to the general method described, from 1.71 g 4h, 0.80 g LiAlH₄, 0.56 mL H₂SO₄, 20 mL plus 10 mL THF, 3.3 mL IPA and 2.5 mL NaOH 2M. Hydrochloride salt formation according to the general method described. Yield: 1.27 g (72.2%) product as a white solid. ¹H-NMR (D₂O): 1.21 (d, MeCH), 2.82 (d, ArCH₂), 3.55 (m, CHNH₃⁺), 3.79 (s, 2 MeO), 6.60 (s, 2 arom. H). ¹⁹F-NMR (D₂O): −99.8 (d), −121.3 (d).

4-(2,2-Difluoro-1-(trideuteromethyl)vinyloxy)-3,5-dimethoxyphenethylamine hydrochloride (Trideutero-DFIPRE; Trideuterodifluoroisopropenylscaline), 5i. According to the general method described, from 1.89 g 3i, 0.88 g LiAlH₄, 0.61 mL H₂SO₄, 20 mL plus 10 mL THF, 3.6 mL IPA and 2.8 mL NaOH 2M. Hydrochloride salt formation according to the general method described. Yield: 1.28 g (65.9%) product as a white solid. ¹H-NMR (D₂O): 2.89 (t, ArCH₂), 3.20 (t, CH₂NH₃⁺), 3.78 (s, 2 MeO), 6.64 (s, 2 arom. H). ¹⁹F-NMR (D₂O): −104.5 (d), −119.3 (d).

4-(2,2-Difluoro-1-(trideuteromethyl)vinyloxy)-3,5-dimethoxyamphetamine hydrochloride (Trideutero-3C-DFIPRE), 6i. According to the general method described, from 1.24 g 4i, 0.55 g LiAlH₄, 0.39 mL H₂SO₄, 15 mL plus 8 mL THF, 2.3 mL IPA and 1.7 mL NaOH 2M. Hydrochloride salt formation according to the general method described. Yield: 0.73 g (57.3%) product as a white solid. ¹H-NMR (D₂O): 1.23 (d, MeCH), 2.83 (d, ArCH₂), 3.56 (m, CHNH₃⁺), 3.78 (s, 2 MeO), 6.61 (s, 2 arom. H). ¹⁹F-NMR (D₂O): −104.5 (d), −119.3 (d).

3,5-Dimethoxy-4-(2-fluoroethoxy)phenethylamine hydrochloride (FE; Fluoroescaline), 5j. According to the general method described, from 4.20 g 3j, 2.63 g LiAlH₄, 1.83 mL H₂SO₄, 70 mL plus 60 mL THF, 11 mL IPA and 8 mL NaOH 2M. Hydrochloride salt formation according to the general method described. Yield: 2.24 g (52.0%) product as a white solid. ¹H-NMR (D₂O): 2.84 (t, ArCH₂), 3.16 (t, CH₂NH₃⁺), 3.74 (s, 2 MeO), 4.11 (dt, ³J(H,F)=32 Hz, CH₂O), 4.58 (dt, ²J(H,F)=48 Hz, CH₂F), 6.58 (s, 2 arom. H).

3,5-Dimethoxy-4-(2-fluoroethoxy)amphetamine hydrochloride (3C-FE), 6j. According to the general method described, from 5.3 g 4j, 2.65 g LiAlH₄, 1.85 mL H₂SO₄, 60 mL plus 30 mL THF, 11 mL IPA and 8.4 mL NaOH 2M. Hydrochloride salt formation according to the general method described. Yield: 4.01 g (73%) product as a white solid. ¹H-NMR (D₂O): 1.19 (d, MeCH), 2.78 (d, ArCH₂), 3.53 (m, CHNH₃⁺), 3.75 (s, 2 MeO), 4.12 (dt, ³J(H,F)=32 Hz, CH₂O), 4.59 (dt, ²J(H,F)=48 Hz, CH₂F), 6.56 (s, 2 arom. H).

4-(2,2-Difluoroethoxy)-3,5-dimethoxyphenethylamine hydrochloride (DFE; Difluoroescaline), 5k. According to the general method described, from 4.10 g 3k, 2.0 g LiAlH₄, 1.40 mL H₂SO₄, 50 mL plus 20 mL THF, 8.4 mL IPA and 6.4 mL NaOH 2M. Hydrochloride salt formation according to the general method described. Yield: 2.39 g (57%) product as a white solid. ¹H-NMR (D₂O): 2.85 (t, ArCH₂), 3.16 (t, CH₂NH₃⁺), 3.76 (s, 2 MeO), 4.10 (dt, ³J(H,F)=15 Hz, CH₂O), 6.05 (tt, ²J(H,F)=55 Hz, CHF₂), 6.59 (s, 2 arom. H).

4-(2,2-Difluoroethoxy)-3,5-dimethoxyamphetamine hydrochloride (3C-DFE), 6k. According to the general method described, from 5.45 g 4k, 2.54 g LiAlH₄, 1.78 mL H₂SO₄, 55 mL plus 30 mL THF, 10.6 mL IPA and 8.1 mL NaOH 2M. Hydrochloride salt formation according to the general method described. Yield: 4.07 g (73%) product as a white solid. ¹H-NMR (D₂O): 1.19 (d, MeCH), 2.79 (d, ArCH₂), 3.53 (m, CHNH₃⁺), 3.76 (s, 2 MeO), 4.10 (dt, ³J(H,F)=15 Hz, CH₂O), 6.05 (tt, ²J(H,F)=55 Hz, CHF₂), 6.56 (s, 2 arom. H).

3,5-Dimethoxy-4-(3-fluoropropoxy)phenethylamine hydrochloride (FP; Fluoroproscaline), 5l. According to the general method described, from 2.35 g 31, 1.17 g LiAlH₄, 0.81 mL H₂SO₄, 25 mL plus 15 mL THF, 4.8 mL IPA and 3.7 mL NaOH 2M. Hydrochloride salt formation according to the general method described. Yield: 1.42 g (59%) product as a white solid. ¹H-NMR (D₂O): 1.99 (dm, ³J(H,F)=26 Hz, CH₂CH₂O), 2.85 (t, ArCH₂), 3.16 (t, CH₂NH₃⁺), 3.76 (s, 2 MeO), 3.98 (t, CH₂O), 4.60 (dt, ²J(H,F)=47 Hz, FCH₂), 6.59 (s, 2 arom. H).

3,5-Dimethoxy-4-(3-fluoropropoxy)amphetamine hydrochloride (3C-FP), 61. According to the general method described, from 3.3 g 41, 1.60 g LiAlH₄, 1.1 mL H₂SO₄, 35 mL plus 15 mL THF, 6.50 mL IPA and 5.0 mL NaOH 2M. Hydrochloride salt formation according to the general method described. Yield: 2.74 g (81%) product as a white solid. ¹H-NMR (D₂O): 1.20 (d, MeCH), 2.00 (dm, ³J(H,F)=27 Hz, CH₂CH₂O), 2.79 (d, ArCH₂), 3.53 (m, CHNH₃⁺), 3.76 (s, 2 MeO), 3.99 (t, CH₂O), 4.60 (dt, ²J(H,F)=47 Hz, FCH₂), 6.57 (s, 2 arom. H).

3,5-Dimethoxy-4-(3-isobutoxy)phenethylamine hydrochloride (IB; Isobuscaline), 5m. According to the general method described, from 2.10 g 3m, 1.06 g LiAlH₄, 0.74 mL H₂SO₄, 25 mL plus 10 mL THF, 4.4 mL IPA and 3.4 mL NaOH 2M. Hydrochloride salt formation according to the general method described. Yield: 1.18 g (55%) product as a white solid. ¹H-NMR (D₂O): 0.86 (d, Me₂CH), 1.88 (m, Me₂CH), 2.85 (t, ArCH₂), 3.17 (t, CH₂NH₃⁺), 3.63 (d, CH₂O), 3.75 (s, 2 MeO), 6.59 (s, 2 arom. H).

3,5-Dimethoxy-4-(3-isobutoxy)amphetamine hydrochloride (3C-IB), 6m. According to the general method described, from 4.05 g 4m, 1.94 g LiAlH₄, 1.36 mL H₂SO₄, 45 mL plus 20 mL THF, 8.1 mL IPA and 6.1 mL NaOH 2M. Hydrochloride salt formation according to the general method described. Yield: 2.5 g (60%) product as a white solid. ¹H-NMR (D₂O): 0.86 (d, Me₂CH—), 1.20 (d, MeCH), 1.90 (m, Me₂CH), 2.79 (d, ArCH₂), 3.54 (m, CHNH₃⁺), 3.65 (d, CH₂O), 3.76 (s, 2 MeO), 6.56 (s, 2 arom. H).

3,5-Dimethoxy-4-propoxyamphetamine hydrochloride (3C-P), 6n. According to the general method described, from 4.70 g 4n, 2.36 g LiAlH₄, 1.65 mL H₂SO₄, 50 mL plus 20 mL THF, 9.8 mL IPA and 7.5 mL NaOH 2M. Hydrochloride salt formation according to the general method described. Yield: 3.27 g (68%) product as a white solid. ¹H-NMR (D₂O): 0.84 (t, MeCH₂), 1.19 (d, MeCH), 1.60 (m, MeCH₂—), 2.78 (d, ArCH₂), 3.52 (m, CHNH₃⁺), 3.74 (s, 2 MeO), 3.81 (t, CH₂O—), 6.56 (s, 2 arom. H).

4-Allyloxy-3,5-dimethoxyamphetamine hydrochloride (3C-AL), 6o. According to the general method described, from 5.13 g 4o, 2.71 g LiAlH₄, 1.90 mL H₂SO₄, 60 mL plus 25 mL THF, 11.3 mL IPA and 8.6 mL NaOH 2M. Hydrochloride salt formation according to the general method described. Yield: 3.44 g (62%) product as a white solid. ¹H-NMR (D₂O): 1.19 (d, MeCH), 2.78 (d, ArCH₂), 3.53 (m, CHNH₃⁺), 3.75 (s, 2 MeO), 4.38 (d, CH₂O—), 5.12-5.24 (m, H₂C═C), 5.93 (m, H₂C═CH), 6.55 (s, 2 arom. H).

3,5-Dimethoxy-4-isopropoxyamphetamine hydrochloride (3C-IP), 6p. According to the general method described, from 4.70 g 4p, 2.36 g LiAlH₄, 1.65 mL H₂SO₄, 50 mL plus 20 mL THF, 9.8 mL IPA and 7.5 mL NaOH 2M. Hydrochloride salt formation according to the general method described. Yield: 3.5 g (72%) product as a white solid. ¹H-NMR (D₂O): 1.12 (d, Me₂CH—), 1.20 (d, MeCH), 2.78 (d, ArCH₂), 3.53 (m, CHNH₃⁺), 3.73 (s, 2 MeO), 4.31 (m, CHO), 6.55 (s, 2 arom. H).

3,5-Dimethoxy-4-methallyloxyamphetamine hydrochloride (3C-MAL), 6q. According to the general method described, from 3.20 g 4q, 1.54 g LiAlH₄, 1.08 mL H₂SO₄, 40 mL plus 15 mL THF, 6.4 mL IPA and 4.9 mL NaOH 2M. Hydrochloride salt formation according to the general method described. Yield: 2.08 g (63%) product as a white solid. ¹H-NMR (D₂O): 1.19 (d, MeCH), 1.73 (s, MeC═), 2.78 (d, ArCH₂), 3.52 (m, CHNH₃⁺), 3.74 (s, 2 MeO), 4.30 (s, CH₂O), 4.88 (m, H₂C═C), 6.55 (s, 2 arom. H).

4-(1,3-Difluoroprop-2-yloxy)-3,5-dimethoxyphenethylamine hydrochloride (DFIP; Difluoroisoproscaline), 5r. According to the general method described, from 0.61 g 3r, 0.34 g LiAlH₄, 0.24 mL H₂SO₄, 12 mL plus 3 mL THF, 1.5 mL IPA and 1.0 mL NaOH 2M. Hydrochloride salt formation according to the general method described. Yield: 0.37 g (59%) product as a white solid. ¹H-NMR (D₂O): 2.92 (t, ArCH₂), 3.23 (t, CH₂NH₃⁺), 3.81 (s, 2 MeO), 4.49 (m, (FCH₂)₂CH), 4.67 (dm, (FCH₂)₂CH), 6.65 (s, 2 arom. H).

3,5-Dimethoxy-4-(1,1,1-trifluoroprop-3-yloxy)phenethylamine hydrochloride (TFP; Trifluoroproscaline), 5s. According to the general method described, from 1.05 g 3s, 0.55 g LiAlH₄, 0.39 mL H₂SO₄, 15 mL plus 5 mL THF, 2.4 mL IPA and 1.7 mL NaOH 2M. Hydrochloride salt formation according to the general method described. Yield: 0.58 g (54%) product as a white solid. ¹H-NMR (D₂O): 2.59 (m, CF₃CH₂), 2.89 (t, ArCH₂), 3.22 (t, CH₂NH₃⁺), 3.79 (s, 2 MeO), 4.09 (t, OCH₂), 6.62 (s, 2 arom. H).

3,5-Dimethoxy-4-vinyloxyphenethylamine hydrogensulfate (V; Viscaline), 5t. According to the general method described, from 3.65 g 3t, 2.48 g LiAlH₄, 1.71 mL H₂SO₄, 75 mL plus 20 mL THF, 10.6 mL IPA and 7.3 mL NaOH 2M. There were obtained 2.24 g (69%) of viscaline as free base. An aliquote (0.24 g) was dissolved in 10 mL anh. diethyl ether and neutralized by careful addition of an 1% H₂SO₄ solution in tetrahydrofuran (prepared from 95-98% sulfuric acid) until the pH value was still slight basic. The mixture was diluted with another 10 mL of diethyl ether, and the white suspension was filtered off, rinsed with diethyl ether and dried in vacuo. Yield: 0.22 g (78%) product as a white solid. ¹H-NMR (DMSO-d₆): 2.79 (t, ArCH₂), 2.98 (t, CH₂NH₃⁺), 3.74 (s, 2 MeO), 4.07 (d, 1H, H₂C═C), 4.15 (d, 1H, H₂C═C), 6.48 (dd, H₂C═CH), 6.62 (s, 2 arom. H).

4-Difluoromethoxy-3,5-dimethoxyphenethylamine hydrochloride (DFM; Difluoromescaline), 5u. According to the general method described, from 0.95 g 3u, 0.59 g LiAlH₄, 0.41 mL H₂SO₄, 15 mL plus 5 mL THF, 2.5 mL IPA and 1.7 mL NaOH 2M. Hydrochloride salt formation according to the general method described. Yield: 0.56 g (57%) product as a white solid. ¹H-NMR (D₂O): 2.84 (t, ArCH₂), 3.14 (t, CH₂NH₃⁺), 3.73 (s, 2 MeO), 6.56 (t, ²J(H,F)=75.6 Hz, F₂CH), 6.59 (s, 2 arom. H).

4-Difluoromethoxy-3,5-dimethoxyamphetamine hydrochloride (3C-DFM), 6u. According to the general method described, from 0.85 g 4u, 0.50 g LiAlH₄, 0.35 mL H₂SO₄, 10 mL plus 5 mL THF, 2.1 mL IPA and 1.5 mL NaOH 2M. Hydrochloride salt formation according to the general method described. Yield: 0.75 g (73%) product as a white solid. ¹H-NMR (D₂O): 1.25 (d, MeCH), 2.87 (m, ArCH₂), 3.59 (m, CHNH₃⁺), 3.82 (s, 2 MeO), 6.66 (t, ²J(H,F)=74.0 Hz, F₂CH), 6.66 (superimposed, s, 2 arom. H).

3,5-Dimethoxy-4-trifluoromethoxyphenethylamine hydrochloride (TFM; Trifluoromescaline), 5v. According to the general method described, from 1.29 g 3v, 0.75 g LiAlH₄, 0.52 mL H₂SO₄, 20 mL plus 8 mL THF, 3.2 mL IPA and 2.2 mL NaOH 2M. Hydrochloride salt formation according to the general method described. Yield: 0.56 g (42%) product as a white solid. ¹H-NMR (D₂O): 2.93 (t, ArCH₂), 3.24 (t, CH₂NH₃⁺), 3.82 (s, 2 MeO), 6.68 (s, 2 arom. H). ¹⁹F-NMR (D₂O): −58.5 (s).

Preparation of Cycloproscaline (14) Via the Simmons-Smith Cyclopropanation

N-BOC-3,5-Dimethoxy-4-vinyloxyphenethylamine, 12. To a solution of 2.0 g (8.96 mmol) viscaline (5t) and 1.27 mL (9.13 mmol) NEt₃ in 15 mL DCM anh. was added dropwise a solution of 2.0 g (9.13 mmol; 1.02 eq) BOC₂O in 10 mL DCM under nitrogen. After stirring for 2 h the mixture was washed with water (2×), HCl 0.25M (2×), NaHCO₃ sat (1×) and brine (1×), dried over MgSO₄ and concentrated in vacuo to get 2.87 g (99%) product as an orange viscous oil. ¹H-NMR (CDCl₃): 1.46 (s, Me₃C), 2.80 (t, ArCH₂), 3.40 (m, CH₂NH), 3.86 (s, 2 MeO), 4.18 (d, 1H, H₂C═C), 4.38 (dd, 1H, H₂C═C), 4.59 (s, NH), 6.46 (s, 2 arom. H), 6.56 (dd, H₂C═CH).

N-BOC-4-Cyclopropoxy-3,5-dimethoxyphenethylamine, 13. To a solution of 20 mL DCM anh. were added 17.32 mL (17.32 mL) Et₂Zn (1M in hexanes) under nitrogen. This solution was cooled using an ice bath and then a solution of 1.33 mL (17.32 mmol) TFA in 10 mL DCM was added over a course of 15 min. After stirring for 30 min, a solution of 1.39 mL CH₂I₂ in 10 mL DCM was added within 3 min. The clear solution was stirred for another 20 min and then a solution of 2.80 g (8.66 mmol) of the vinyl ether 12 in 10 mL DCM was added during 5 min and the ice bath was removed. After 30 min the reaction mixture was cooled again using an ice bath and 3.0 mL NEt₃ were added before saturated NaHCO₃ was added and the mixture was stirred vigorously for 10 min. The solids were removed by filtration. The two layers of the filtrate were separated, and the org. layer was washed with water (4×), dried over MgSO₄ and concentrated in vacuo. The residue was dissolved in a small amount of diethyl ether and filtered and rinsed through a small pad of silica gel to remove any zinc salts. After evaporation in vacuo there were obtained 2.39 g (82%) product as an orange sticky oil. ¹H-NMR (CDCl₃): 0.49 (m, CH₂CH), 0.93 (m, CH₂CH), 1.47 (s, Me₃C), 2.77 (t, ArCH₂), 3.42 (m, CH₂NH), 3.89 (s, 2 MeO), 4.16 (m, CHO), 4.56 (s, NH), 6.42 (s, 2 arom. H).

4-Cyclopropoxy-3,5-dimethoxyphenethylamine hydrochloride (CP; Cycloproscaline), 14. A solution of 2.35 g (6.96 mmol) N-BOC-4-Cyclopropoxy-3,5-dimethoxyphenethylamine (13) in 20 mL dioxane anh. was treated with 8 mL 4M HCl anh. in dioxane under nitrogen. The mixture was allowed to stir overnight. Next, the volatiles were stripped off in vacuo, and the residue was dissolved in a small amount of iPrOH and treated with EtOAc under stirring. The solids formed were filtered off and rinsed with additional EtOAc and diethyl ether. Yield after drying: 1.04 g (63%) product as a white solid. ¹H-NMR (D₂O): 0.47 (m, CH₂CH), 0.77 (m, CH₂CH), 2.91 (t, ArCH₂), 3.22 (t, CH₂NH₃⁺), 3.80 (s, 2 MeO), 4.12 (m, CHO), 6.65 (s, 2 arom. H).

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible considering the above teachings. It is, therefore, to be understood that within the scope of the appended claims, the invention can be practiced otherwise than as specifically described.

### REFERENCES

1. Andersson M, Persson M, & Kjellgren A (2017). Psychoactive substances as a last resort—a qualitative study of self-treatment of migraine and cluster headaches. Harm Reduct J 14: 60.
2. Basel GfCIi (1932). Verfahren zur Darstellung eines Derivates des Phenyläthylamins. In Eigenösisches Amt für Geistiges Eigentum. ed. Basel G.f.C.I.: Schweiz.
3. Bogenschutz M P (2013). Studying the effects of classic hallucinogens in the treatment of alcoholism: rationale, methodology, and current research with psilocybin. Curr Drug Abuse Rev 6: 17-29.
4. Bogenschutz M P, Forcehimes A A, Pommy J A, Wilcox C E, Barbosa P C, & Strassman R J (2015). Psilocybin-assisted treatment for alcohol dependence: a proof-of-concept study. J Psychopharmacol 29: 289-299.
5. Carhart-Harris R L, Bolstridge M, Rucker J, Day C M, Erritzoe D, Kaelen M, Bloomfield M, Rickard J A, Forbes B, Feilding A, Taylor D, Pilling S, Curran V H, & Nutt D J (2016a). Psilocybin with psychological support for treatment-resistant depression: an open-label feasibility study. Lancet Psychiatry 3: 619-627.
6. Carhart-Harris R L, Kaelen M, Bolstridge M, Williams T M, Williams L T, Underwood R, Feilding A, & Nutt D J (2016b). The paradoxical psychological effects of lysergic acid diethylamide (LSD). Psychol Med 46: 1379-1390.
7. Cassels B, & Saez-Briones P (2018). Dark classics in chemical neuroscience: mescaline. ACS Chem Neurosci in press.
8. Charalampous W, Kinross-Wright (1966). Metabolic Fate of Mescaline in Man. Psychopharmacologia 9: 48-63.
9. Davis A K, Barrett F S, May D G, Cosimano M P, Sepeda N D, Johnson M W, Finan P H, & Griffiths R R (2020). Effects of Psilocybin-Assisted Therapy on Major Depressive Disorder: A Randomized Clinical Trial. JAMA Psychiatry.
10. de Araujo D B Acute, lasting and antidepressant effects of Ayahuasca.
11. Dolder P C, Schmid Y, Mueller F, Borgwardt S, & Liechti M E (2016). LSD acutely impairs fear recognition and enhances emotional empathy and sociality. Neuropsychopharmacology 41: 2638-2646.
12. Dolder P C, Schmid Y, Steuer A E, Kraemer T, Rentsch K M, Hammann F, & Liechti M E (2017). Pharmacokinetics and pharmacodynamics of lysergic acid diethylamide in healthy subjects. Clin Pharmacokinetics 56: 1219-1230.
13. Dominguez-Clave E, Soler J, Elices M, Pascual J C, Alvarez E, de la Fuente Revenga M, Friedlander P, Feilding A, & Riba J (2016). Ayahuasca: Pharmacology, neuroscience and therapeutic potential. Brain Res Bull 126: 89-101.
14. Dos Santos R G, Osorio F L, Crippa J A, Riba J, Zuardi A W, & Hallak J E (2016). Antidepressive, anxiolytic, and antiaddictive effects of ayahuasca, psilocybin and lysergic acid diethylamide (LSD): a systematic review of clinical trials published in the last 25 years. Ther Adv Psychopharmacol 6: 193-213.
15. Garcia-Romeu A, Davis A K, Erowid F, Erowid E, Griffiths R R, & Johnson M W (2019). Cessation and reduction in alcohol consumption and misuse after psychedelic use. J Psychopharmacol: 269881119845793.
16. Garcia-Romeu A, Griffiths R R, & Johnson M W (2015). Psilocybin-occasioned mystical experiences in the treatment of tobacco addiction. Curr Drug Abuse Rev 7: 157-164.
17. Gasser P, Holstein D, Michel Y, Doblin R, Yazar-Klosinski B, Passie T, & Brenneisen R (2014). Safety and efficacy of lysergic acid diethylamide-assisted psychotherapy for anxiety associated with life-threatening diseases. J Nerv Ment Dis 202: 513-520.
18. Gasser P, Kirchner K, & Passie T (2015). LSD-assisted psychotherapy for anxiety associated with a life-threatening disease: a qualitative study of acute and sustained subjective effects. J Psychopharmacol 29: 57-68.
19. Griffiths R, Richards W, Johnson M, McCann U, & Jesse R (2008). Mystical-type experiences occasioned by psilocybin mediate the attribution of personal meaning and spiritual significance 14 months later. J Psychopharmacol 22: 621-632.
20. Griffiths R R, Johnson M W, Carducci M A, Umbricht A, Richards W A, Richards B D, Cosimano M P, & Klinedinst M A (2016). Psilocybin produces substantial and sustained decreases in depression and anxiety in patients with life-threatening cancer: a randomized double-blind trial. J Psychopharmacol 30: 1181-1197.
21. Grob C S, Danforth A L, Chopra G S, Hagerty M, McKay C R, Halberstadt A L, & Greer G R (2011). Pilot study of psilocybin treatment for anxiety in patients with advanced-stage cancer. Arch Gen Psychiatry 68: 71-78.
22. Holze F, Duthaler U, Vizeli P, Muller F, Borgwardt S, & Liechti M E (2019). Pharmacokinetics and subjective effects of a novel oral LSD formulation in healthy subjects. Br J Clin Pharmacol 85: 1474-1483.
23. Holze F, Vizeli P, Ley L, Müller F, Dolder P, Stocker M, Duthaler U, Varghese N, Eckert A, Borgwardt S, & Liechti M E (2020). Acute dose-dependent effects of lysergic acid diethylamide in a double-blind placebo-controlled study in healthy subjects. Neuropsychopharmacology doi: 10.1038/s41386-020-00883-6.
24. Hysek C M, Simmler L D, Nicola V, Vischer N, Donzelli M, Krahenbihl S, Grouzmann E, Hoener M C, & Liechti M E (2012). Duloxetine inhibits effects of MDMA (“ecstasy”) in vitro and in humans in a randomized placebo-controlled laboratory study. PLoS One 7: e36476.
25. Ichikawa J, & Meltzer H Y (2000). The effect of serotonin(1A) receptor agonism on antipsychotic drug-induced dopamine release in rat striatum and nucleus accumbens. Brain Res 858: 252-263.
26. Johnson M W, Garcia-Romeu A, Cosimano M P, & Griffiths R R (2014). Pilot study of the 5-HT2AR agonist psilocybin in the treatment of tobacco addiction. J Psychopharmacol 28: 983-992.
27. Johnson M W, Garcia-Romeu A, & Griffiths R R (2016). Long-term follow-up of psilocybin-facilitated smoking cessation. Am J Drug Alcohol Abuse 43: 55-60.
28. Kolaczynska K, Luethi D, Trachsel D, Hoener M C, & Liechti M E (2022). Receptor interaction profiles of 4-alkoxy-3,5-dimethoxyphenethylamines (mescaline derivatives) and related amphetamines. Front Pharmacol doi: 10.3389/fphar.2021.794254.
29. Krebs T S, & Johansen P O (2012). Lysergic acid diethylamide (LSD) for alcoholism: meta-analysis of randomized controlled trials. J Psychopharmacol 26: 994-1002.
30. Liechti M E (2017). Modern clinical research on LSD. Neuropsychopharmacology 42: 2114-2127.
31. Luethi D, & Liechti M E (2018). Monoamine transporter and receptor interaction profiles in vitro predict reported human doses of novel psychoactive stimulants and psychedelics. Int J Neuropsychopharmacol 21: 926-931.
32. Madsen M K, Fisher P M, Burmester D, Dyssegaard A, Stenbaek D S, Kristiansen S, Johansen S S, Lehel S, Linnet K, Svarer C, Erritzoe D, Ozenne B, & Knudsen G M (2019). Correction: Psychedelic effects of psilocybin correlate with serotonin 2A receptor occupancy and plasma psilocin levels. Neuropsychopharmacology.
33. Marona-Lewicka D, Thisted R A, & Nichols D E (2005). Distinct temporal phases in the behavioral pharmacology of LSD: dopamine D2 receptor-mediated effects in the rat and implications for psychosis. Psychopharmacology (Berl) 180: 427-435.
34. Matsuya Y, Sasaki K, Ochiai H, & Nemoto H (2007). Synthesis and biological evaluation of dihydrofuran-fused perhydrophenanthrenes as a new anti-influenza agent having novel structural characteristic. Bioorg Med Chem 15: 424-432.
35. McKinley N F, & O'Shea D F (2004). Efficient synthesis of aryl vinyl ethers exploiting 2,4,6-trivinylcyclotriboroxane as a vinylboronic acid equivalent. J Org Chem 69: 5087-5092.
36. Nakai T, Tanaka K, & Ishikawa N (1976). Reactions of 2,2,2,-trifluoroethoxy- and 2,2,2-trifluoroethylthiobenzene with lithium dialkylamides: the formation of phenylthioynamines. Chem Letters 5: 1263-1266.
37. O'Shea P D, Chen C Y, Chen W, Dagneau P, Frey L F, Grabowski E J, Marcantonio K M, Reamer R A, Tan L, Tillyer R D, Roy A, Wang X, & Zhao D (2005). Practical asymmetric synthesis of a potent PDE4 inhibitor via stereoselective enolate alkylation of a chiral aryl-heteroaryl secondary tosylate. J Org Chem 70: 3021-3030.
38. Palhano-Fontes F, Barreto D, Onias H, Andrade K C, Novaes M M, Pessoa J A, Mota-Rolim S A, Osorio F L, Sanches R, Dos Santos R G, Tofoli L F, de Oliveira Silveira G, Yonamine M, Riba J, Santos F R, Silva-Junior A A, Alchieri J C, Galvao-Coelho N L, Lobao-Soares B, Hallak J E C, Arcoverde E, Maia-de-Oliveira J P, & Araujo D B (2019). Rapid antidepressant effects of the psychedelic ayahuasca in treatment-resistant depression: a randomized placebo-controlled trial. Psychol Med 49: 655-663.
39. Passie T, Halpern J H, Stichtenoth D O, Emrich H M, & Hintzen A (2008). The pharmacology of lysergic acid diethylamide: a review. CNS Neurosci Ther 14: 295-314.
40. Rickli A, Moning O D, Hoener M C, & Liechti M E (2016). Receptor interaction profiles of novel psychoactive tryptamines compared with classic hallucinogens. Eur Neuropsychopharmacol 26: 1327-1337.
41. Roseman L, Nutt D J, & Carhart-Harris R L (2017). Quality of acute psychedelic experience predicts therapeutic efficacy of psilocybin for treatment-resistant depression. Front Pharmacol 8: 974.
42. Ross S, Bossis A, Guss J, Agin-Liebes G, Malone T, Cohen B, Mennenga S E, Belser A, Kalliontzi K, Babb J, Su Z, Corby P, & Schmidt B L (2016). Rapid and sustained symptom reduction following psilocybin treatment for anxiety and depression in patients with life-threatening cancer: a randomized controlled trial. J Psychopharmacol 30: 1165-1180.
43. Sanches R F, de Lima Osorio F, Dos Santos R G, Macedo L R, Maia-de-Oliveira J P, Wichert-Ana L, de Araujo D B, Riba J, Crippa J A, & Hallak J E (2016). Antidepressant Effects of a Single Dose of Ayahuasca in Patients With Recurrent Depression: A SPECT Study. J Clin Psychopharmacol 36: 77-81.
44. Schmid Y, Enzler F, Gasser P, Grouzmann E, Preller K H, Vollenweider F X, Brenneisen R, Mueller F, Borgwardt S, & Liechti M E (2015). Acute effects of lysergic acid diethylamide in healthy subjects. Biol Psychiatry 78: 544-553.
45. Schmid Y, Gasser P, Oehen P, & Liechti M E (2020). Acute subjective effects in LSD- and MDMA-assisted psychotherapy. J Psychopharmacol.
46. Schmid Y, & Liechti M E (2018). Long-lasting subjective effects of LSD in normal subjects. Psychopharmacology (Berl) 235: 535-545.
47. Shulgin A, & Shulgin A (1991) PiHKAL: a chemical love story. 1 edn.
48. Trachsel D (2002). Synthese von neuen (Phenylalkyl)aminen zur Untersuchung von Struktur-Aktivitatsbeziehungen, Mitteilung 1: Mescalin Derivative. Helvetica Chimica Acta 85: 3019-3026.
49. Trachsel D, Lehmann D, & Enzensperger C (2013) Phenethylemine: Von der Struktur zur Funktion. 1 edn.: Solothurn: Nachtschatten Verlag.
50. Vollenweider F X, Vontobel P, Hell D, & Leenders K L (1999). 5-HT modulation of dopamine release in basal ganglia in psilocybin-induced psychosis in man—a PET study with [11C]raclopride. Neuropsychopharmacology 20: 424-433.

What is claimed is:

1. A composition comprising a compound represented by FIG. 1, which is
characterized in that R is one of the following substituents: hydrogen, methyl, or ethyl, and further characterized in that R′ is one of the following substituents
C₁-C₅ branched or unbranched alkyl with the alkyl optionally substituted with F₁-F₅ fluorine substituents up to a fully fluorinated alkyl, or
C₃-C₆ cycloalkyl optionally and independently substituted with one or more substituents such as F₁-F₅ fluorine and/or C₁-C₂ alkyl, or
(C₃-C₆ cycloalkyl)-C₁-C₂ branched or unbranched alkyl optionally substituted with one or more substituents such as F₁-F₅ fluorine and/or C₁-C₂ alkyl, or
C₂-C₅ branched or unbranched alkenyl with E or Z vinylic, cis or trans allylic, E or Z allylic or other double bond position in relation to the attached ether function, where any of the carbons of the branched or unbranched alkenyl substituent is optionally substituted independently with one or more C₁-C₂ alkyl, with F₁-F₅ fluorine or with D₁-D₅ deuteron substituents.

2. The composition of claim 1, further characterized in that the compound is a free base.

3. The composition of claim 1, further characterized in that the compound is a salt thereof.

4. The composition of claim 3, further characterized in that the compound is a hydrochloride salt thereof.

5. The composition of claim 4, further characterized in that the compound is a pharmacologically acceptable acid addition salt thereof.

6. The compounds of claim 1, further characterized in that the compound is chosen from the group consisting of a racemate, a single enantiomer, a diastereomer, and a mixture of enantiomers or diastereomers in any ratio.

7. A method of changing neurotransmission, including the steps of:
administering a pharmaceutically effective amount of composition to a mammal of a compound represented by FIG. 1, which is characterized in that R is one of the following substituents: hydrogen, methyl, or ethyl, and which is further characterized in that R′ is one of:
C₁-C₅ branched or unbranched alkyl with the alkyl optionally substituted with F₁-F₅ fluorine substituents up to a fully fluorinated alkyl, or
C₃-C₆ cycloalkyl optionally and independently substituted with one or more substituents such as F₁-F₅ fluorine and/or C₁-C₂ alkyl, or
(C₃-C₆ cycloalkyl)-C₁-C₂ branched or unbranched alkyl optionally substituted with one or more substituents such as F₁-F₅ fluorine and/or C₁-C₂ alkyl, or
C₂-C₅ branched or unbranched alkenyl with E or Z vinylic, cis or trans allylic, E or Z allylic or other double bond position in relation to the attached ether function, where any of the carbons of the branched or unbranched alkenyl substituent is optionally substituted independently with one or more C₁-C₂ alkyl, with F₁-F₅ fluorine or with D₁-D₅ deuteron substituents;
increasing serotonin 5-HT2A and 5-HT2C receptor interaction in the mammal; and
inducing psychoactive effects.

8. The method of claim 7, wherein the compound is chosen from the group consisting of a racemate, a single enantiomer, a single diastereomer, and a mixture of enantiomers or diastereomers in any ratio.

9. The method of claim 7, wherein the psychoactive effects include psychedelic or empathogenic effects having intensity, effect quality, or duration of effect in a mammal in comparison to that of mescaline.

10. The method of claim 7, wherein the compound is administered to mammals for substance-assisted psychotherapy.

11. The method of claim 7, wherein the compound is administered to allow for changing dose potency in comparison to mescaline.

12. The method of claim 7, wherein the compound is administered to allow for tailoring and treatment individualization to the mammal's therapeutic need.

13. The method of claim 7, wherein the mammal is a human.

14. A method of deuteration to obtain a compound represented by FIG. 1, which is
characterized in that R is one of the following substituents: hydrogen, methyl, or ethyl, and which is further characterized in that R′ is one of the following substituents
C₁-C₅ branched or unbranched alkyl with the alkyl optionally substituted with F₁-F₅ fluorine substituents up to a fully fluorinated alkyl, or
C₃-C₆ cycloalkyl optionally and independently substituted with one or more substituents such as F₁-F₅ fluorine and/or C₁-C₂ alkyl, or
(C₃-C₆ cycloalkyl)-C₁-C₂ branched or unbranched alkyl optionally substituted with one or more substituents such as F₁-F₅ fluorine and/or C₁-C₂ alkyl, or
C₂-C₅ branched or unbranched alkenyl with E or Z vinylic, cis or trans allylic, E or Z allylic or other double bond position in relation to the attached ether function, where any of the carbons of the branched or unbranched alkenyl substituent is optionally substituted independently with one or more C₁-C₂ alkyl, with F₁-F₅ fluorine or with D₁-D₅ deuteron substituents,
consisting of the steps of:
abstracting protons from a reacting molecule and its intermediates;
covalently binding these initially abstracted protons in-situ; and
quenching the resulting metalated difluorovinyl ether with a deuterium source.

15. The method of claim 14, wherein the reacting molecule is compound 7 and the intermediate is compound 10a.

16. The method of claim 14, wherein said abstracting protons step is achieved by adding a deprotonating agent.

17. The method of claim 16, wherein the deprotonating agent is chosen from the group consisting of diisopropylamide, tert-butoxide, bis(trimethylsilyl)amide, and tetramethylpiperidides.

18. The method of claim 17, wherein the deprotonating agent is a tetramethylpiperidide and is chosen from the group of tetramethylpiperidides of lithium, sodium, and potassium.

19. The method of claim 14, wherein said covalently binding step is achieved by adding a reagent chosen from the group consisting of butyl lithium and methyl lithium.

20. The method of claim 14, wherein the deuterium source is chosen from the group consisting of D2O and a deuterated alcohol.
